SEQUENCE LISTING (231 sequences)

SEQ ID NO: 1 - protein, 113 aa, Artificial Sequence
  synthetic sequence
EVQLVESGGGLVQPGGSLRLSCAASGFTFSNYWMNWVRQAPGKGLEWVADIRLKSNNYATHYAESVKGRFTISRDNAKNSLYLQMNSLRAEDTAVYYCVRDGPYWGQGTLVTV

SEQ ID NO: 2 - protein, 125 aa, Artificial Sequence
  synthetic sequence
EVQLVESGGGLVQPGGSLRLSCAASGFTFSSYWMSWVRQAPGKGLEWVANIKQDGSEKYYVDSVKGRFTISRDNAKNSLYLQMNSLRAEDTAVYYCVRDGPYWGQGTLVTVSAASTKGPSVFPLA

SEQ ID NO: 3 - protein, 113 aa, Artificial Sequence
  synthetic sequence
DVVMTQSPLSLPVTLGQPASISCRSSQSLVYSDGNTYLNWFQQRPGQSPRRLIYKVSNRDSGVPDRFSGSGSGTDFTLKISRVEAEDVGVYYCFQGSHVPRTFGGGTKLEIKR

SEQ ID NO: 4 - protein, 113 aa, Artificial Sequence
  synthetic sequence
DVVMTQSPLSLPVTLGQPASISCRSSQSIVHSNGNTYLEWFQQRPGQSPRRLIYKISNRFSGVPDRFSGSGSGTDFTLKISRVEAEDVGVYYCFQGSHVPRTFGGGTKLEIKR

SEQ ID NO: 5 - protein, 120 aa, Artificial Sequence
  synthetic sequence
QVQLVQSGAEVKKPGASVKVSCKASGYTFTSYWMHWVRQAPGQGLEWMGTIYPRNSDTDYNQKFKARVTMTRDTSTSTVYMELSSLRSEDTAVYYCARPLYYYVTSPPTLLWGQGTLVTV

SEQ ID NO: 6 - protein, 120 aa, Artificial Sequence
  synthetic sequence
QVQLVQSGAEVKKPGASVKVSCKASGYIFTSYWMHWVRQAPGQGLEWMGTIYPRNSDTDYNQKFKARVTMTRDTSTSTVYMELSSLRSEDTAVYYCARPLYYYVTSPPTLLWGQGTLVTV

SEQ ID NO: 7 - protein, 120 aa, Artificial Sequence
  synthetic sequence
QVQLVQSGAEVKKPGASVKVSCKASGYNFTSYWMHWVRQAPGQGLEWMGTIYPRNSDTDYNQKFKARVTMTRDTSTSTVYMELSSLRSEDTAVYYCARPLYYYVTSPPTLLWGQGTLVTV

SEQ ID NO: 8 - protein, 120 aa, Artificial Sequence
  synthetic sequence
QVQLVQSGAEVKKPGASVKVSCKASGYSFTSYWMHWVRQAPGQGLEWMGTIYPRNSDTDYNQKFKARVTMTRDTSTSTVYMELSSLRSEDTAVYYCARPLYYYVTSPPTLLWGQGTLVTV

SEQ ID NO: 9 - protein, 120 aa, Artificial Sequence
  synthetic sequence
QVQLVQSGAEVKKPGASVKVSCKASGYTFHSYWMHWVRQAPGQGLEWMGTIYPRNSDTDYNQKFKARVTMTRDTSTSTVYMELSSLRSEDTAVYYCARPLYYYVTSPPTLLWGQGTLVTV

SEQ ID NO: 10 - protein, 120 aa, Artificial Sequence
  synthetic sequence
QVQLVQSGAEVKKPGASVKVSCKASGYTFSSYWMHWVRQAPGQGLEWMGTIYPRNSDTDYNQKFKARVTMTRDTSTSTVYMELSSLRSEDTAVYYCARPLYYYVTSPPTLLWGQGTLVTV

SEQ ID NO: 11 - protein, 120 aa, Artificial Sequence
  synthetic sequence
QVQLVQSGAEVKKPGASVKVSCKASGYTFTAYWMHWVRQAPGQGLEWMGTIYPRNSDTDYNQKFKARVTMTRDTSTSTVYMELSSLRSEDTAVYYCARPLYYYVTSPPTLLWGQGTLVTV

SEQ ID NO: 12 - protein, 120 aa, Artificial Sequence
  synthetic sequence
QVQLVQSGAEVKKPGASVKVSCKASGYTFTGYWMHWVRQAPGQGLEWMGTIYPRNSDTDYNQKFKARVTMTRDTSTSTVYMELSSLRSEDTAVYYCARPLYYYVTSPPTLLWGQGTLVTV

SEQ ID NO: 13 - protein, 120 aa, Artificial Sequence
  synthetic sequence
QVQLVQSGAEVKKPGASVKVSCKASGYTFTNYWMHWVRQAPGQGLEWMGTIYPRNSDTDYNQKFKARVTMTRDTSTSTVYMELSSLRSEDTAVYYCARPLYYYVTSPPTLLWGQGTLVTV

SEQ ID NO: 14 - protein, 120 aa, Artificial Sequence
  synthetic sequence
QVQLVQSGAEVKKPGASVKVSCKASGYTFTTYWMHWVRQAPGQGLEWMGTIYPRNSDTDYNQKFKARVTMTRDTSTSTVYMELSSLRSEDTAVYYCARPLYYYVTSPPTLLWGQGTLVTV

SEQ ID NO: 15 - protein, 120 aa, Artificial Sequence
  synthetic sequence
QVQLVQSGAEVKKPGASVKVSCKASGYTFTSFWMHWVRQAPGQGLEWMGTIYPRNSDTDYNQKFKARVTMTRDTSTSTVYMELSSLRSEDTAVYYCARPLYYYVTSPPTLLWGQGTLVTV

SEQ ID NO: 16 - protein, 120 aa, Artificial Sequence
  synthetic sequence
QVQLVQSGAEVKKPGASVKVSCKASGYTFTSHWMHWVRQAPGQGLEWMGTIYPRNSDTDYNQKFKARVTMTRDTSTSTVYMELSSLRSEDTAVYYCARPLYYYVTSPPTLLWGQGTLVTV

SEQ ID NO: 17 - protein, 120 aa, Artificial Sequence
  synthetic sequence
QVQLVQSGAEVKKPGASVKVSCKASGYTFTSYWMHWVRQAPGQHLEWMGTIYPRNSDTDYNQKFKARVTMTRDTSTSTVYMELSSLRSEDTAVYYCARPLYYYVTSPPTLLWGQGTLVTV

SEQ ID NO: 18 - protein, 120 aa, Artificial Sequence
  synthetic sequence
QVQLVQSGAEVKKPGASVKVSCKASGYTFTSYWMHWVRQAPGQGLEWMGTFYPRNSDTDYNQKFKARVTMTRDTSTSTVYMELSSLRSEDTAVYYCARPLYYYVTSPPTLLWGQGTLVTV

SEQ ID NO: 19 - protein, 120 aa, Artificial Sequence
  synthetic sequence
QVQLVQSGAEVKKPGASVKVSCKASGYTFTSYWMHWVRQAPGQGLEWMGTLYPRNSDTDYNQKFKARVTMTRDTSTSTVYMELSSLRSEDTAVYYCARPLYYYVTSPPTLLWGQGTLVTV

SEQ ID NO: 20 - protein, 120 aa, Artificial Sequence
  synthetic sequence
QVQLVQSGAEVKKPGASVKVSCKASGYTFTSYWMHWVRQAPGQGLEWMGTNYPRNSDTDYNQKFKARVTMTRDTSTSTVYMELSSLRSEDTAVYYCARPLYYYVTSPPTLLWGQGTLVTV

SEQ ID NO: 21 - protein, 120 aa, Artificial Sequence
  synthetic sequence
QVQLVQSGAEVKKPGASVKVSCKASGYTFTSYWMHWVRQAPGQGLEWMGTVYPRNSDTDYNQKFKARVTMTRDTSTSTVYMELSSLRSEDTAVYYCARPLYYYVTSPPTLLWGQGTLVTV

SEQ ID NO: 22 - protein, 120 aa, Artificial Sequence
  synthetic sequence
QVQLVQSGAEVKKPGASVKVSCKASGYTFTSYWMHWVRQAPGQGLEWMGTINPRNSDTDYNQKFKARVTMTRDTSTSTVYMELSSLRSEDTAVYYCARPLYYYVTSPPTLLWGQGTLVTV

SEQ ID NO: 23 - protein, 120 aa, Artificial Sequence
  synthetic sequence
QVQLVQSGAEVKKPGASVKVSCKASGYTFTSYWMHWVRQAPGQGLEWMGTITPRNSDTDYNQKFKARVTMTRDTSTSTVYMELSSLRSEDTAVYYCARPLYYYVTSPPTLLWGQGTLVTV

SEQ ID NO: 24 - protein, 120 aa, Artificial Sequence
  synthetic sequence
QVQLVQSGAEVKKPGASVKVSCKASGYTFTSYWMHWVRQAPGQGLEWMGTIYPRNSDIDYNQKFKARVTMTRDTSTSTVYMELSSLRSEDTAVYYCARPLYYYVTSPPTLLWGQGTLVTV

SEQ ID NO: 25 - protein, 120 aa, Artificial Sequence
  synthetic sequence
QVQLVQSGAEVKKPGASVKVSCKASGYTFTSYWMHWVRQAPGQGLEWMGTIYPRNSDTDYNQNFKARVTMTRDTSTSTVYMELSSLRSEDTAVYYCARPLYYYVTSPPTLLWGQGTLVTV

SEQ ID NO: 26 - protein, 120 aa, Artificial Sequence
  synthetic sequence
QVQLVQSGAEVKKPGASVKVSCKASGYTFTSYWMHWVRQAPGQGLEWMGTIYPRNSDTDYNQRFKARVTMTRDTSTSTVYMELSSLRSEDTAVYYCARPLYYYVTSPPTLLWGQGTLVTV

SEQ ID NO: 27 - protein, 120 aa, Artificial Sequence
  synthetic sequence
QVQLVQSGAEVKKPGASVKVSCKASGYTFTSYWMHWVRQAPGQGLEWMGTIYPRNSDTDYNQKFKARVTVTRDTSTSTVYMELSSLRSEDTAVYYCARPLYYYVTSPPTLLWGQGTLVTV

SEQ ID NO: 28 - protein, 120 aa, Artificial Sequence
  synthetic sequence
QVQLVQSGAEVKKPGASVKVSCKASGYTFTSYWMHWVRQAPGQGLEWMGTIYPRNSDTDYNQKFKARVTMSRDTSTSTVYMELSSLRSEDTAVYYCARPLYYYVTSPPTLLWGQGTLVTV

SEQ ID NO: 29 - protein, 120 aa, Artificial Sequence
  synthetic sequence
QVQLVQSGAEVKKPGASVKVSCKASGYTFTSYWMHWVRQAPGQGLEWMGTIYPRNSDTDYNQKFKARVTMTRDTSTNTVYMELSSLRSEDTAVYYCARPLYYYVTSPPTLLWGQGTLVTV

SEQ ID NO: 30 - protein, 120 aa, Artificial Sequence
  synthetic sequence
QVQLVQSGAEVKKPGASVKVSCKASGYTFTSYWMHWVRQAPGQGLEWMGTIYPRNSDTDYNQKFKARVTMTRDTSTTTVYMELSSLRSEDTAVYYCARPLYYYVTSPPTLLWGQGTLVTV

SEQ ID NO: 31 - protein, 120 aa, Artificial Sequence
  synthetic sequence
QVQLVQSGAEVKKPGASVKVSCKASGYTFTSYWMHWVRQAPGQGLEWMGTIYPRNSDTDYNQKFKARVTMTRDTSTSTVYMDLSSLRSEDTAVYYCARPLYYYVTSPPTLLWGQGTLVTV

SEQ ID NO: 32 - protein, 120 aa, Artificial Sequence
  synthetic sequence
QVQLVQSGAEVKKPGASVKVSCKASGYTFTSYWMHWVRQAPGQGLEWMGTIYPRNSDTDYNQKFKARVTMTRDTSTSTVYMELRSLRSEDTAVYYCARPLYYYVTSPPTLLWGQGTLVTV

SEQ ID NO: 33 - protein, 120 aa, Artificial Sequence
  synthetic sequence
QVQLVQSGAEVKKPGASVKVSCKASGYTFTSYWMHWVRQAPGQGLEWMGTIYPRNSDTDYNQKFKARVTMTRDTSTSTVYMELSSLTSEDTAVYYCARPLYYYVTSPPTLLWGQGTLVTV

SEQ ID NO: 34 - protein, 120 aa, Artificial Sequence
  synthetic sequence
QVQLVQSGAEVKKPGASVKVSCKASGYTFTSYWMHWVRQAPGQGLEWMGTIYPRNSDTDYNQKFKARVTMTRDTSTSTAYMELSSLRSEDTAVYYCARPLYYYVTSPPTLLWGQGTLVTV

SEQ ID NO: 35 - protein, 120 aa, Artificial Sequence
  synthetic sequence
QVQLVQSGAEVKKPGASVKVSCKASGYTFTSYWMHWVRQAPGQGLEWMGTIYPRNSDTDYNQKFKARVTMTRDTSTSTVYMELSSLRSDDTAVYYCARPLYYYVTSPPTLLWGQGTLVTV

SEQ ID NO: 36 - protein, 120 aa, Artificial Sequence
  synthetic sequence
QVQLVQSGAEVKKPGASVKVSCKASGYTFTSYWMHWVRQAPGQGLEWMGTIYPRNSDTDYNQKFKARVTMTRDTSTSTVYMELSSLRSEDTAIYYCARPLYYYVTSPPTLLWGQGTLVTV

SEQ ID NO: 37 - protein, 120 aa, Artificial Sequence
  synthetic sequence
QVQLVQSGAEVKKPGASVKVSCKASGYTFTSYWMHWVRQAPGQGLEWMGTIYPRNSDTDYNQKFKARVTMTRDTSTSIVYMELSSLRSEDTAVYYCARPLYYYVTSPPTLLWGQGTLVTV

SEQ ID NO: 38 - protein, 120 aa, Artificial Sequence
  synthetic sequence
QVQLVQSGAEVKKPGASVKVSCKASGYTFTSYFMHWVRQAPGQGLEWMGTIYPRNSDTDYNQKFKARVTMTRDTSTSTVYMELSSLRSEDTAVYYCARPLYYYVTSPPTLLWGQGTLVTV

SEQ ID NO: 39 - protein, 120 aa, Artificial Sequence
  synthetic sequence
QVQLVQSGAEVKKPGASVKVSCKASGYTFTSYWMHWVRQAPGQGLEWMGTIDPRNSDTDYNQKFKARVTMTRDTSTSTVYMELSSLRSEDTAVYYCARPLYYYVTSPPTLLWGQGTLVTV

SEQ ID NO: 40 - protein, 120 aa, Artificial Sequence
  synthetic sequence
QVQLVQSGAEVKKPGASVKVSCKASGYTFTSYWMHWVRQAPGQGLEWMGTIYPRNSDTDYNQKFKARVTMTRDTSTRTVYMELSSLRSEDTAVYYCARPLYYYVTSPPTLLWGQGTLVTV

SEQ ID NO: 41 - protein, 120 aa, Artificial Sequence
  synthetic sequence
QVQLVESGAEVKKPGASVKVSCKASGYTFTSYWMHWVRQAPGQGLEWMGTIYPRNSDTDYNQKFKARVTMTRDTSTSTVYMELSSLRSEDTAVYYCARPLYYYVTSPPTLLWGQGTLVTV

SEQ ID NO: 42 - protein, 120 aa, Artificial Sequence
  synthetic sequence
QVQLVQSGAEVKKPGASVKVSCKASGYTFTDYWMHWVRQAPGQGLEWMGTIYPRNSDTDYNQKFKARVTMTRDTSTSTVYMELSSLRSEDTAVYYCARPLYYYVTSPPTLLWGQGTLVTV

SEQ ID NO: 43 - protein, 120 aa, Artificial Sequence
  synthetic sequence
QVQLVQSGAEVKKPGASVKVSCKASGYTFTSYWIHWVRQAPGQGLEWMGTIYPRNSDTDYNQKFKARVTMTRDTSTSTVYMELSSLRSEDTAVYYCARPLYYYVTSPPTLLWGQGTLVTV

SEQ ID NO: 44 - protein, 120 aa, Artificial Sequence
  synthetic sequence
QVQLVQSGAEVKKPGASVKVSCKASGYTFTSYWLHWVRQAPGQGLEWMGTIYPRNSDTDYNQKFKARVTMTRDTSTSTVYMELSSLRSEDTAVYYCARPLYYYVTSPPTLLWGQGTLVTV

SEQ ID NO: 45 - protein, 120 aa, Artificial Sequence
  synthetic sequence
QVQLVQSGAEVKKPGASVKVSCKASGYTFTSYWMNWVRQAPGQGLEWMGTIYPRNSDTDYNQKFKARVTMTRDTSTSTVYMELSSLRSEDTAVYYCARPLYYYVTSPPTLLWGQGTLVTV

SEQ ID NO: 46 - protein, 120 aa, Artificial Sequence
  synthetic sequence
QVQLVQSGAEVKKPGASVKVSCKASGYTFTSYWMHWVRQAPGQGLEWMGTIYPRNSDTDFNQKFKARVTMTRDTSTSTVYMELSSLRSEDTAVYYCARPLYYYVTSPPTLLWGQGTLVTV

SEQ ID NO: 47 - protein, 120 aa, Artificial Sequence
  synthetic sequence
QVQLVQSGAEVKKPGASVKVSCKASGYTFTSYWMHWVRQAPGQGLEWMGTIYPRNSDTDHNQKFKARVTMTRDTSTSTVYMELSSLRSEDTAVYYCARPLYYYVTSPPTLLWGQGTLVTV

SEQ ID NO: 48 - protein, 120 aa, Artificial Sequence
  synthetic sequence
QVQLVQSGAEVKKPGASVKVSCKASGYTFTSYWMHWVRQAPGQGLEWMGTIYPRNSDTDYNQKFKARVTMTRDTSTSTTYMELSSLRSEDTAVYYCARPLYYYVTSPPTLLWGQGTLVTV

SEQ ID NO: 49 - protein, 120 aa, Artificial Sequence
  synthetic sequence
QVQLVQSGAEVKKPGASVKVSCKASGYTFTSHWMHWVRQAPGQGLEWMGTIYPRNSDTDYNQKFKARVTMTRDTSTSIVYMELSSLRSEDTAVYYCARPLYYYVTSPPTLLWGQGTLVTV

SEQ ID NO: 50 - protein, 122 aa, Artificial Sequence
  synthetic sequence
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARPLYYYVTSPPTLLWGQGTLVTVSS

SEQ ID NO: 51 - protein, 108 aa, Artificial Sequence
  synthetic sequence
DIQLTQSPSFLSASVGDRVTITCKASQDVGTAVAWYQQKPGKAPKLLIYWASTRHTGVPSRFSGSGSGTEFTLTISSLQPEDFATYYCQQCNSYPFTFGSGTKLEIKR

SEQ ID NO: 52 - protein, 108 aa, Artificial Sequence
  synthetic sequence
DIQLTQSPSFLSASVGDRVTITCKASQDVGTAVAWYQQKPGKAPKLLIYWASTRHTGVPSRFSGSGSGTEFTLTISSLQPEDFATYYCQQVNSYPFTFGSGTKLEIKR

SEQ ID NO: 53 - protein, 108 aa, Artificial Sequence
  synthetic sequence
DIQLTQSPSFLSASVGDRVTITCKASQDVGTAVAWYQQKPGKAPKLLIYWASTRHTGVPSRFSGSGSGTEFTLTISSLQPEDFATYYCQQINSYPFTFGSGTKLEIKR

SEQ ID NO: 54 - protein, 108 aa, Artificial Sequence
  synthetic sequence
DIQLTQSPSFLSASVGDRVTITCKASQDVGTAVAWYQQKPGKAPKLLIYWASTRHTGVPSRFSGSGSGTEFTLTISSLQPEDFATYYCQQLNSYPFTFGSGTKLEIKR

SEQ ID NO: 55 - protein, 108 aa, Artificial Sequence
  synthetic sequence
DIQLTQSPSFLSASVGDRVTITCKASQDVGTAVAWYQQKPGKAPKLLIYWASTRHTGVPSRFSGSGSGTEFTLTISSLQPEDFATYYCQQANSYPFTFGSGTKLEIKR

SEQ ID NO: 56 - protein, 108 aa, Artificial Sequence
  synthetic sequence
DIQLTQSPSFLSASVGDRVTITCKASQDVGTAVAWYQQKPGKAPKLLIYWASTRHTGVPSRFSGSGSGTEFTLTISSLQPEDFATYYCQQSNSYPFTFGSGTKLEIKR

SEQ ID NO: 57 - protein, 108 aa, Artificial Sequence
  synthetic sequence
DIQLTQSPSFLSASVGDRVTITCKASQDVGTAVAWYQQKPGKAPKLLIYWASTRHTGVPSRFSGSGSGTEFTLTISSLQPEDFATYYCQQASTYPFTFGSGTKLEIKR

SEQ ID NO: 58 - protein, 108 aa, Artificial Sequence
  synthetic sequence
DIQLTQSPSFLSASVGDRVTITCKASQDVGTAVAWYQQKPGKAPKLLIYWASTRHTGVPSRFSGSGSGTEFTLTISSLQPEDFATYYCQQASAYPFTFGSGTKLEIKR

SEQ ID NO: 59 - protein, 108 aa, Artificial Sequence
  synthetic sequence
DIQLTQSPSFLSASVGDRVTITCKASQDVGTAVAWYQQKPGKAPKLLIYWASTRHTGVPSRFSGSGSGTEFTLTISSLQPEDFATYYCQQARTYPFTFGSGTKLEIKR

SEQ ID NO: 60 - protein, 108 aa, Artificial Sequence
  synthetic sequence
DIQLTQSPSFLSASVGDRVTITCKASQDVGTAVAWYQQKPGKAPKLLIYWASTRHTGVPSRFSGSGSGTEFTLTISSLQPEDFATYYCQQARAYPFTFGSGTKLEIKR

SEQ ID NO: 61 - protein, 108 aa, Artificial Sequence
  synthetic sequence
DIQLTQSPSFLSASVGDRVTITCKASQDVGTAVAWYQQKPGKAPKLLIYWASTRHTGVPSRFSGSGSGTEFTLTISSLQPEDFATYYCQQAKTYPFTFGSGTKLEIKR

SEQ ID NO: 62 - protein, 108 aa, Artificial Sequence
  synthetic sequence
DIQLTQSPSFLSASVGDRVTITCKASQDVGTAVAWYQQKPGKAPKLLIYWASTRHTGVPSRFSGSGSGTEFTLTISSLQPEDFATYYCQQAAAYPFTFGSGTKLEIKR

SEQ ID NO: 63 - protein, 108 aa, Artificial Sequence
  synthetic sequence
DIQLTQSPSFLSASVGDRVTITCKASQDVGTAVAWYQQKPGKAPKLLIYWASTRHTGVPSRFSGSGSGTEFTLTISSLQPEDFATYYCQQAQTYPFTFGSGTKLEIKR

SEQ ID NO: 64 - protein, 108 aa, Artificial Sequence
  synthetic sequence
DIQLTQSPSFLSASVGDRVTITCKASQDVGTAVAWYQQKPGKAPKLLIYWASTRHTGVPSRFSGSGSGTEFTLTISSLQPEDFATYYCQQAQAYPFTFGSGTKLEIKR

SEQ ID NO: 65 - protein, 108 aa, Artificial Sequence
  synthetic sequence
DIQLTQSPSFLSASVGDRVTITCKASQDVGTAVAWYQQKPGKAPKLLIYWASTRHTGVPSRFSGSGSGTEFTLTISSLQPEDFATYYCQQAYSYPFTFGSGTKLEIKR

SEQ ID NO: 66 - protein, 108 aa, Artificial Sequence
  synthetic sequence
DIQLTQSPSFLSASVGDRVTITCRASQGISSYLAWYQQKPGKAPKLLIYAASTLQSGVPSRFSGSGSGTEFTLTISSLQPEDFATYYCQQCNSYPFTFGSGTKLEIKR

SEQ ID NO: 67 - protein, 139 aa, Artificial Sequence
  synthetic sequence
MRAWIFFLLCLAGRALAQVQLVQSGAEVKKPGASVKVSCKASGYTFTSYWMHWVRQAPGQGLEWMGTIYPRNSDTDYNQKFKARVTMTRDTSTSTVYMELSSLRSEDTAVYYCARPLYYYVTSPPTLLWGQGTLVTVSS

SEQ ID NO: 68 - protein, 139 aa, Artificial Sequence; identical to SEQ ID NO: 67
  synthetic sequence

SEQ ID NO: 69 - protein, 139 aa, Artificial Sequence
  synthetic sequence
MRAWIFFLLCLAGRALAQVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARPLYYYVTSPPTLLWGQGTLVTVSS

SEQ ID NO: 70 - protein, 139 aa, Artificial Sequence
  synthetic sequence
MRAWIFFLLCLAGRALAQVQLVQSGAEVKKPGASVKVSCKASGYTFTSYWMHWVRQAPGQGLEWMGTIYPRNSDTDYNQKFKARVTMTRDTSTSTAYMELSSLRSEDTAVYYCARPLYYYVTSPPTLLWGQGTLVTVSS

SEQ ID NO: 71 - protein, 139 aa, Artificial Sequence
  synthetic sequence
MRAWIFFLLCLAGRALAQVQLVQSGAEVKKPGASVKVSCKASGYTFTSYWMHWVRQAPGQGLEWMGTIDPRNSDTDYNQKFKARVTMTRDTSTSTVYMELSSLRSEDTAVYYCARPLYYYVTSPPTLLWGQGTLVTVSS

SEQ ID NO: 72 - protein, 139 aa, Artificial Sequence
  synthetic sequence
MRAWIFFLLCLAGRALAQVQLVQSGAEVKKPGASVKVSCKASGYTFTSYWMHWVRQAPGQGLEWMGTITPRNSDTDYNQKFKARVTMTRDTSTSTVYMELSSLRSEDTAVYYCARPLYYYVTSPPTLLWGQGTLVTVSS

SEQ ID NO: 73 - protein, 139 aa, Artificial Sequence
  synthetic sequence
MRAWIFFLLCLAGRALAQVQLVQSGAEVKKPGASVKVSCKASGYTFTSYWMHWVRQAPGQGLEWMGTINPRNSDTDYNQKFKARVTMTRDTSTSTVYMELSSLRSEDTAVYYCARPLYYYVTSPPTLLWGQGTLVTVSS

SEQ ID NO: 74 - protein, 139 aa, Artificial Sequence
  synthetic sequence
MRAWIFFLLCLAGRALAQVQLVQSGAEVKKPGASVKVSCKASGYTFTSYWMHWVRQAPGQGLEWMGTIYPRNSDTDYNQKFKARVTMTRDTSTSTVYMDLSSLRSEDTAVYYCARPLYYYVTSPPTLLWGQGTLVTVSS

SEQ ID NO: 75 - protein, 139 aa, Artificial Sequence
  synthetic sequence
MRAWIFFLLCLAGRALAQVQLVQSGAEVKKPGASVKVSCKASGYTFTSYWMHWVRQAPGQGLEWMGTIYPRNSDTDYNQKFKARVTMTRDTSTSTVYMELSSLRSDDTAVYYCARPLYYYVTSPPTLLWGQGTLVTVSS

SEQ ID NO: 76 - protein, 139 aa, Artificial Sequence
  synthetic sequence
MRAWIFFLLCLAGRALAQVQLVQSGAEVKKPGASVKVSCKASGYTFTSYFMHWVRQAPGQGLEWMGTIYPRNSDTDYNQKFKARVTMTRDTSTSTVYMELSSLRSEDTAVYYCARPLYYYVTSPPTLLWGQGTLVTVSS

SEQ ID NO: 77 - protein, 139 aa, Artificial Sequence
  synthetic sequence
MRAWIFFLLCLAGRALAQVQLVQSGAEVKKPGASVKVSCKASGYTFTGYWMHWVRQAPGQGLEWMGTIYPRNSDTDYNQKFKARVTMTRDTSTSTVYMELSSLRSEDTAVYYCARPLYYYVTSPPTLLWGQGTLVTVSS

SEQ ID NO: 78 - protein, 139 aa, Artificial Sequence
  synthetic sequence
MRAWIFFLLCLAGRALAQVQLVQSGAEVKKPGASVKVSCKASGYTFTAYWMHWVRQAPGQGLEWMGTIYPRNSDTDYNQKFKARVTMTRDTSTSTVYMELSSLRSEDTAVYYCARPLYYYVTSPPTLLWGQGTLVTVSS

SEQ ID NO: 79 - protein, 139 aa, Artificial Sequence
  synthetic sequence
MRAWIFFLLCLAGRALAQVQLVQSGAEVKKPGASVKVSCKASGYTFTNYWMHWVRQAPGQGLEWMGTIYPRNSDTDYNQKFKARVTMTRDTSTSTVYMELSSLRSEDTAVYYCARPLYYYVTSPPTLLWGQGTLVTVSS

SEQ ID NO: 80 - protein, 139 aa, Artificial Sequence
  synthetic sequence
MRAWIFFLLCLAGRALAQVQLVQSGAEVKKPGASVKVSCKASGYTFTTYWMHWVRQAPGQGLEWMGTIYPRNSDTDYNQKFKARVTMTRDTSTSTVYMELSSLRSEDTAVYYCARPLYYYVTSPPTLLWGQGTLVTVSS

SEQ ID NO: 81 - protein, 139 aa, Artificial Sequence
  synthetic sequence
MRAWIFFLLCLAGRALAQVQLVQSGAEVKKPGASVKVSCKASGYTFTSHWMHWVRQAPGQGLEWMGTIYPRNSDTDYNQKFKARVTMTRDTSTSTVYMELSSLRSEDTAVYYCARPLYYYVTSPPTLLWGQGTLVTVSS

SEQ ID NO: 82 - protein, 139 aa, Artificial Sequence
  synthetic sequence
MRAWIFFLLCLAGRALAQVQLVQSGAEVKKPGASVKVSCKASGYTFTSYWMHWVRQAPGQHLEWMGTIYPRNSDTDYNQKFKARVTMTRDTSTSTVYMELSSLRSEDTAVYYCARPLYYYVTSPPTLLWGQGTLVTVSS

SEQ ID NO: 83 - protein, 139 aa, Artificial Sequence
  synthetic sequence
MRAWIFFLLCLAGRALAQVQLVQSGAEVKKPGASVKVSCKASGYTFTSYWMHWVRQAPGQGLEWMGTIYPRNSDTDYNQKFKARVTMTRDTSTSTVYMELSSLRSEDTAIYYCARPLYYYVTSPPTLLWGQGTLVTVSS

SEQ ID NO: 84 - protein, 139 aa, Artificial Sequence
  synthetic sequence
MRAWIFFLLCLAGRALAQVQLVQSGAEVKKPGASVKVSCKASGYTFTSYWMHWVRQAPGQGLEWMGTIYPRNSDTDYNQKFKARVTMTRDTSTNTVYMELSSLRSEDTAVYYCARPLYYYVTSPPTLLWGQGTLVTVSS

SEQ ID NO: 85 - protein, 139 aa, Artificial Sequence
  synthetic sequence
MRAWIFFLLCLAGRALAQVQLVQSGAEVKKPGASVKVSCKASGYTFTSYWMHWVRQAPGQGLEWMGTIYPRNSDTDYNQKFKARVTMTRDTSTTTVYMELSSLRSEDTAVYYCARPLYYYVTSPPTLLWGQGTLVTVSS

SEQ ID NO: 86 - protein, 139 aa, Artificial Sequence
  synthetic sequence
MRAWIFFLLCLAGRALAQVQLVQSGAEVKKPGASVKVSCKASGYTFTSYWMHWVRQAPGQGLEWMGTIYPRNSDTDYNQKFKARVTMTRDTSTRTVYMELSSLRSEDTAVYYCARPLYYYVTSPPTLLWGQGTLVTVSS

SEQ ID NO: 87 - protein, 139 aa, Artificial Sequence
  synthetic sequence
MRAWIFFLLCLAGRALAQVQLVQSGAEVKKPGASVKVSCKASGYTFSSYWMHWVRQAPGQGLEWMGTIYPRNSDTDYNQKFKARVTMTRDTSTSTVYMELSSLRSEDTAVYYCARPLYYYVTSPPTLLWGQGTLVTVSS

SEQ ID NO: 88 - protein, 139 aa, Artificial Sequence
  synthetic sequence
MRAWIFFLLCLAGRALAQVQLVQSGAEVKKPGASVKVSCKASGYTFHSYWMHWVRQAPGQGLEWMGTIYPRNSDTDYNQKFKARVTMTRDTSTSTVYMELSSLRSEDTAVYYCARPLYYYVTSPPTLLWGQGTLVTVSS

SEQ ID NO: 89 - protein, 139 aa, Artificial Sequence
  synthetic sequence
MRAWIFFLLCLAGRALAQVQLVQSGAEVKKPGASVKVSCKASGYTFTSYWMHWVRQAPGQGLEWMGTIYPRNSDTDYNQKFKARVTMSRDTSTSTVYMELSSLRSEDTAVYYCARPLYYYVTSPPTLLWGQGTLVTVSS

SEQ ID NO: 90 - protein, 139 aa, Artificial Sequence
  synthetic sequence
MRAWIFFLLCLAGRALAQVQLVQSGAEVKKPGASVKVSCKASGYTFTSYWMHWVRQAPGQGLEWMGTIYPRNSDTDYNQKFKARVTMTRDTSTSTVYMELSSLTSEDTAVYYCARPLYYYVTSPPTLLWGQGTLVTVSS

SEQ ID NO: 91 - protein, 139 aa, Artificial Sequence
  synthetic sequence
MRAWIFFLLCLAGRALAQVQLVQSGAEVKKPGASVKVSCKASGYTFTSYWMHWVRQAPGQGLEWMGTIYPRNSDTDYNQKFKARVTVTRDTSTSTVYMELSSLRSEDTAVYYCARPLYYYVTSPPTLLWGQGTLVTVSS

SEQ ID NO: 92 - protein, 139 aa, Artificial Sequence
  synthetic sequence
MRAWIFFLLCLAGRALAQVQLVQSGAEVKKPGASVKVSCKASGYNFTSYWMHWVRQAPGQGLEWMGTIYPRNSDTDYNQKFKARVTMTRDTSTSTVYMELSSLRSEDTAVYYCARPLYYYVTSPPTLLWGQGTLVTVSS

SEQ ID NO: 93 - protein, 139 aa, Artificial Sequence
  synthetic sequence
MRAWIFFLLCLAGRALAQVQLVQSGAEVKKPGASVKVSCKASGYIFTSYWMHWVRQAPGQGLEWMGTIYPRNSDTDYNQKFKARVTMTRDTSTSTVYMELSSLRSEDTAVYYCARPLYYYVTSPPTLLWGQGTLVTVSS

SEQ ID NO: 94 - protein, 139 aa, Artificial Sequence
  synthetic sequence
MRAWIFFLLCLAGRALAQVQLVQSGAEVKKPGASVKVSCKASGYSFTSYWMHWVRQAPGQGLEWMGTIYPRNSDTDYNQKFKARVTMTRDTSTSTVYMELSSLRSEDTAVYYCARPLYYYVTSPPTLLWGQGTLVTVSS

SEQ ID NO: 95 - protein, 139 aa, Artificial Sequence
  synthetic sequence
MRAWIFFLLCLAGRALAQVQLVQSGAEVKKPGASVKVSCKASGYTFTSFWMHWVRQAPGQGLEWMGTIYPRNSDTDYNQKFKARVTMTRDTSTSTVYMELSSLRSEDTAVYYCARPLYYYVTSPPTLLWGQGTLVTVSS

SEQ ID NO: 96 - protein, 139 aa, Artificial Sequence
  synthetic sequence
MRAWIFFLLCLAGRALAQVQLVQSGAEVKKPGASVKVSCKASGYTFTSYWMHWVRQAPGQGLEWMGTNYPRNSDTDYNQKFKARVTMTRDTSTSTVYMELSSLRSEDTAVYYCARPLYYYVTSPPTLLWGQGTLVTVSS

SEQ ID NO: 97 - protein, 139 aa, Artificial Sequence
  synthetic sequence
MRAWIFFLLCLAGRALAQVQLVQSGAEVKKPGASVKVSCKASGYTFTSYWMHWVRQAPGQGLEWMGTLYPRNSDTDYNQKFKARVTMTRDTSTSTVYMELSSLRSEDTAVYYCARPLYYYVTSPPTLLWGQGTLVTVSS

SEQ ID NO: 98 - protein, 139 aa, Artificial Sequence
  synthetic sequence
MRAWIFFLLCLAGRALAQVQLVQSGAEVKKPGASVKVSCKASGYTFTSYWMHWVRQAPGQGLEWMGTVYPRNSDTDYNQKFKARVTMTRDTSTSTVYMELSSLRSEDTAVYYCARPLYYYVTSPPTLLWGQGTLVTVSS

SEQ ID NO: 99 - protein, 139 aa, Artificial Sequence
  synthetic sequence
MRAWIFFLLCLAGRALAQVQLVQSGAEVKKPGASVKVSCKASGYTFTSYWMHWVRQAPGQGLEWMGTFYPRNSDTDYNQKFKARVTMTRDTSTSTVYMELSSLRSEDTAVYYCARPLYYYVTSPPTLLWGQGTLVTVSS

SEQ ID NO: 100 - protein, 139 aa, Artificial Sequence
  synthetic sequence
MRAWIFFLLCLAGRALAQVQLVQSGAEVKKPGASVKVSCKASGYTFTSYWMHWVRQAPGQGLEWMGTIYPRNSDIDYNQKFKARVTMTRDTSTSTVYMELSSLRSEDTAVYYCARPLYYYVTSPPTLLWGQGTLVTVSS

SEQ ID NO: 101 - protein, 139 aa, Artificial Sequence
  synthetic sequence
MRAWIFFLLCLAGRALAQVQLVQSGAEVKKPGASVKVSCKASGYTFTSYWMHWVRQAPGQGLEWMGTIYPRNSDTDYNQRFKARVTMTRDTSTSTVYMELSSLRSEDTAVYYCARPLYYYVTSPPTLLWGQGTLVTVSS

SEQ ID NO: 102 - protein, 139 aa, Artificial Sequence
  synthetic sequence
MRAWIFFLLCLAGRALAQVQLVQSGAEVKKPGASVKVSCKASGYTFTSYWMHWVRQAPGQGLEWMGTIYPRNSDTDYNQNFKARVTMTRDTSTSTVYMELSSLRSEDTAVYYCARPLYYYVTSPPTLLWGQGTLVTVSS

SEQ ID NO: 103 - protein, 139 aa, Artificial Sequence
  synthetic sequence
MRAWIFFLLCLAGRALAQVQLVQSGAEVKKPGASVKVSCKASGYTFTSYWMHWVRQAPGQGLEWMGTIYPRNSDTDYNQKFKARVTMTRDTSTSTVYMELRSLRSEDTAVYYCARPLYYYVTSPPTLLWGQGTLVTVSS

SEQ ID NO: 104 - protein, 139 aa, Artificial Sequence
  synthetic sequence
MRAWIFFLLCLAGRALAQVQLVQSGAEVKKPGASVKVSCKASGYTFTSYWMHWVRQAPGQGLEWMGTIYPRNSDTDYNQKFKARVTMTRDTSTSTTYMELSSLRSEDTAVYYCARPLYYYVTSPPTLLWGQGTLVTVSS

SEQ ID NO: 105 - protein, 139 aa, Artificial Sequence
  synthetic sequence
MRAWIFFLLCLAGRALAQVQLVESGAEVKKPGASVKVSCKASGYTFTSYWMHWVRQAPGQGLEWMGTIYPRNSDTDYNQKFKARVTMTRDTSTSTVYMELSSLRSEDTAVYYCARPLYYYVTSPPTLLWGQGTLVTVSS

SEQ ID NO: 106 - protein, 139 aa, Artificial Sequence
  synthetic sequence
MRAWIFFLLCLAGRALAQVQLVQSGAEVKKPGASVKVSCKASGYTFTDYWMHWVRQAPGQGLEWMGTIYPRNSDTDYNQKFKARVTMTRDTSTSTVYMELSSLRSEDTAVYYCARPLYYYVTSPPTLLWGQGTLVTVSS

SEQ ID NO: 107 - protein, 139 aa, Artificial Sequence
  synthetic sequence
MRAWIFFLLCLAGRALAQVQLVQSGAEVKKPGASVKVSCKASGYTFTSYWIHWVRQAPGQGLEWMGTIYPRNSDTDYNQKFKARVTMTRDTSTSTVYMELSSLRSEDTAVYYCARPLYYYVTSPPTLLWGQGTLVTVSS

SEQ ID NO: 108 - protein, 139 aa, Artificial Sequence
  synthetic sequence
MRAWIFFLLCLAGRALAQVQLVQSGAEVKKPGASVKVSCKASGYTFTSYWLHWVRQAPGQGLEWMGTIYPRNSDTDYNQKFKARVTMTRDTSTSTVYMELSSLRSEDTAVYYCARPLYYYVTSPPTLLWGQGTLVTVSS

SEQ ID NO: 109 - protein, 139 aa, Artificial Sequence
  synthetic sequence
MRAWIFFLLCLAGRALAQVQLVQSGAEVKKPGASVKVSCKASGYTFTSYWMNWVRQAPGQGLEWMGTIYPRNSDTDYNQKFKARVTMTRDTSTSTVYMELSSLRSEDTAVYYCARPLYYYVTSPPTLLWGQGTLVTVSS

SEQ ID NO: 110 - protein, 139 aa, Artificial Sequence
  synthetic sequence
MRAWIFFLLCLAGRALAQVQLVQSGAEVKKPGASVKVSCKASGYTFTSYWMHWVRQAPGQGLEWMGTIYPRNSDTDHNQKFKARVTMTRDTSTSTVYMELSSLRSEDTAVYYCARPLYYYVTSPPTLLWGQGTLVTVSS

SEQ ID NO: 111 - protein, 139 aa, Artificial Sequence
  synthetic sequence
MRAWIFFLLCLAGRALAQVQLVQSGAEVKKPGASVKVSCKASGYTFTSYWMHWVRQAPGQGLEWMGTIYPRNSDTDFNQKFKARVTMTRDTSTSTVYMELSSLRSEDTAVYYCARPLYYYVTSPPTLLWGQGTLVTVSS

SEQ ID NO: 112 - protein, 139 aa, Artificial Sequence
  synthetic sequence
MRAWIFFLLCLAGRALAQVQLVQSGAEVKKPGASVKVSCKASGYTFTSYWMHWVRQAPGQGLEWMGTIYPRNSDTDYNQKFKARVTMTRDTSTSIVYMELSSLRSEDTAVYYCARPLYYYVTSPPTLLWGQGTLVTVSS

SEQ ID NO: 113 - protein, 139 aa, Artificial Sequence
  synthetic sequence
MRAWIFFLLCLAGRALAQVQLVQSGAEVKKPGASVKVSCKASGYTFTSHWMHWVRQAPGQGLEWMGTIYPRNSDTDYNQKFKARVTMTRDTSTSIVYMELSSLRSEDTAVYYCARPLYYYVTSPPTLLWGQGTLVTVSS

SEQ ID NO: 114 - protein, 139 aa, Artificial Sequence
  synthetic sequence
MRAWIFFLLCLAGRALAQVQLVQSGAEVKKPGASVKVSCKASGYTFTSYWMHWVRQAPGQGLEWMGTIYPRNSDTDYNQKFKARVTMTRDTSTSTVYMELSSLRSEDTAVYYCARPLYYYITSPPTLLWGQGTLVTVSS

SEQ ID NO: 115 - protein, 139 aa, Artificial Sequence
  synthetic sequence
MRAWIFFLLCLAGRALAQVQLVQSGAEVKKPGASVKVSCKASGYTFTSYWMHWVRQAPGQGLEWMGTIYPRNSDTDYNQKFKARVTMTRDTSTSTVYMELSSLRSEDTAVYYCARPLYYYLTSPPTLLWGQGTLVTVSS

SEQ ID NO: 116 - protein, 139 aa, Artificial Sequence; identical to SEQ ID NO: 115
  synthetic sequence

SEQ ID NO: 117 - protein, 139 aa, Artificial Sequence
  synthetic sequence
MRAWIFFLLCLAGRALAQVQLVQSGAEVKKPGASVKVSCKASGYTFTSYWMHWVRQAPGQGLEWMGTIYPRNSDTDYNQKFKARVTMTRDTSTSTVYMELSSLRSEDTAVYYCARPLYYYDTSPPTLLWGQGTLVTVSS

SEQ ID NO: 118 - protein, 139 aa, Artificial Sequence
  synthetic sequence
MRAWIFFLLCLAGRALAQVQLVQSGAEVKKPGASVKVSCKASGYTFTSYWMHWVRQAPGQGLEWMGTIYPRNSDTDYNQKFKARVTMTRDTSTSTVYMELSSLRSEDTAVYYCARPLYYYFTSPPTLLWGQGTLVTVSS

SEQ ID NO: 119 - protein, 139 aa, Artificial Sequence
  synthetic sequence
MRAWIFFLLCLAGRALAQVQLVQSGAEVKKPGASVKVSCKASGYTFTSYWMHWVRQAPGQGLEWMGTIYPRNSDTDYNQKFKARVTMTRDTSTSTVYMELSSLRSEDTAVYYCARPLYYYATSPPTLLWGQGTLVTVSS

SEQ ID NO: 120 - protein, 139 aa, Artificial Sequence
  synthetic sequence
MRAWIFFLLCLAGRALAQVQLVQSGAEVKKPGASVKVSCKASGYTFTSYWMHWVRQAPGQGLEWMGTIYPRNSDTDYNQKFKARVTMTRDTSTSTVYMELSSLRSEDTAVYYCARPLYYYGTSPPTLLWGQGTLVTVSS

SEQ ID NO: 121 - protein, 139 aa, Artificial Sequence
  synthetic sequence
MRAWIFFLLCLAGRALAQVQLVQSGAEVKKPGASVKVSCKASGYTFTSYWMHWVRQAPGQGLEWMGTIYPRNSDTDYNQKFKARVTMTRDTSTSTVYMELSSLRSEDTAVYYCARPLYYYVTSPPTLFWGQGTLVTVSS

SEQ ID NO: 122 - protein, 139 aa, Artificial Sequence
  synthetic sequence
MRAWIFFLLCLAGRALAQVQLVQSGAEVKKPGASVKVSCKASGYTFTSYWMHWVRQAPGQGLEWMGTIYPRNSDTDYNQKFKARVTMTRDTSTSTVYMELSSLRSEDTAVYYCARPLYYYVTSPPTLVWGQGTLVTVSS

SEQ ID NO: 123 - protein, 139 aa, Artificial Sequence
  synthetic sequence
MRAWIFFLLCLAGRALAQVQLVQSGAEVKKPGASVKVSCKASGYTFTSYWMHWVRQAPGQGLEWMGTIYPRNSDTDYNQKFKARVTMTRDTSTSTVYMELSSLRSEDTAVYYCARPLYYYVTSPPTLPWGQGTLVTVSS

SEQ ID NO: 124 - protein, 139 aa, Artificial Sequence
  synthetic sequence
MRAWIFFLLCLAGRALAQVQLVQSGAEVKKPGASVKVSCKASGYTFTSYWMHWVRQAPGQGLEWMGTIYPRNSDTDYNQKFKARVTMTRDTSTSTVYMELSSLRSEDTAVYYCARPLYYYLTSPPTLFWGQGTLVTVSS

SEQ ID NO: 125 - protein, 139 aa, Artificial Sequence
  synthetic sequence
MRAWIFFLLCLAGRALAQVQLVQSGAEVKKPGASVKVSCKASGYTFTSYWMHWVRQAPGQGLEWMGTIYPRNSNTDYNQKFKARVTMTRDTSTSTVYMELSSLRSEDTAVYYCARPLYYYVTSPPTLLWGQGTLVTVSS

SEQ ID NO: 126 - protein, 139 aa, Artificial Sequence
  synthetic sequence
MRAWIFFLLCLAGRALAQVQLMQSGAEVKKPGASVKVSCKASGYTFTSYWMHWVRQAPGQGLEWMGTIYPRNSDTDYNQKFKARVTMTRDTSTSTVYMELSSLRSEDTAVYYCARPLYYYVTSPPTLLWGQGTLVTVSS

SEQ ID NO: 127 - protein, 139 aa, Artificial Sequence; identical to SEQ ID NO: 121
  synthetic sequence

SEQ ID NO: 128 - protein, 139 aa, Artificial Sequence
  synthetic sequence
MRAWIFFLLCLAGRALAQVQLVQSGAEVKKPGASVKVSCKASGYTFTSYWMHWVRQAPGQGLEWMGIIYPRNSDTDYNQKFKARVTMTRDTSTSTVYMELSSLRSEDTAVYYCARPLYYYVTSPPTLLWGQGTLVTVSS

SEQ ID NO: 129 - protein, 139 aa, Artificial Sequence
  synthetic sequence
MRAWIFFLLCLAGRALAQVQLVQSGAEVKKPGASVKVSCKASGYTFTSYWMHWVRQAPGQGLEWMGTIYPRNSDTDYNQKFKARVTMTRDTSTSTVYMELSSLRSEDTAVYYCARPLYYYVISPPTLLWGQGTLVTVSS

SEQ ID NO: 130 - protein, 139 aa, Artificial Sequence; identical to SEQ ID NO: 67
  synthetic sequence

SEQ ID NO: 131 - protein, 139 aa, Artificial Sequence
  synthetic sequence
MRAWIFFLLCLAGRALAQVQLMQSGAEVKKPGASVKVSCKASGYTFTSYWMHWVRQAPGQGLEWMGTIYPRNSNTDYNQKFKARVTMTRDTSTSTVYMELSSLRSEDTAVYYCARPLYYYLTSPPTLFWGQGTLVTVSS

SEQ ID NO: 132 - protein, 139 aa, Artificial Sequence
  synthetic sequence
MRAWIFFLLCLAGRALAQVQLVQSGAEVKKPGASVKVSCKASGYTFTSYWMHWMRQAPGQGLEWMGTIYPRNSDTDYNQKFKARVTMTRDTSTSTVYMELSSLRSEDTAVYYCARPLYYYVTSPPTLFWGQGTLVTVSS

SEQ ID NO: 133 - protein, 139 aa, Artificial Sequence
  synthetic sequence
MRAWIFFLLCLAGRALAQVQLVQSGAEVKKPGASVKVSCKASGYTFTSYWMHWMRQAPGQGLEWMGTIYPRNSDTDYNQKFKARVTMTRDTSTSTVYMELSSLRSEDTAVYYCARPLYYYVTSPPTLLWGQGTLVTVSS

SEQ ID NO: 134 - protein, 139 aa, Artificial Sequence
  synthetic sequence
MRAWIFFLLCLAGRALAQVQLMQSGAEVKKPGASVKVSCKASGYTFTSYWMHWVRQAPGQGLEWMGTIYPRNSNTDYNQKFKARVTMTRDTSTSTVYMELSSLRSEDTAVYYCARPLYYYLTSPPTLLWGQGTLVTVSS

SEQ ID NO: 135 - protein, 139 aa, Artificial Sequence
  synthetic sequence
MRAWIFFLLCLAGRALAQVQLMQSGAEVKKPGASVKVSCKASGYTFTSYWMHWVRQAPGQGLEWMGTIYPRNSNTDYNQKFKARVTMTRDTSTSTVYMELSSLRSEDTAVYYCARPLYYYVTSPPTLFWGQGTLVTVSS

SEQ ID NO: 136 - protein, 139 aa, Artificial Sequence; identical to SEQ ID NO: 131
  synthetic sequence

SEQ ID NO: 137 - protein, 139 aa, Artificial Sequence
  synthetic sequence
MRAWIFFLLCLAGRALAQVQLMQSGAEVKKPGASVKVSCKASGYTFTSYWMHWMRQAPGQGLEWMGTIYPRNSNTDYNQKFKARVTMTRDTSTSTVYMELSSLRSEDTAVYYCARPLYYYLTSPPTLFWGQGTLVTVSS

SEQ ID NO: 138 - protein, 139 aa, Artificial Sequence
  synthetic sequence
MRAWIFFLLCLAGRALAQVQLMQSGAEVKKPGASVKVSCKASGYTFTSYWMHWMRQAPGQGLEWMGTIYPRNSNTDYNQKFKARVTMTRDTSTSTVYMELSSLRSEDTAVYYCARPLYYYLTSPPTLLWGQGTLVTVSS

SEQ ID NO: 139 - protein, 139 aa, Artificial Sequence
  synthetic sequence
MRAWIFFLLCLAGRALAQVQLMQSGAEVKKPGASVKVSCKASGYTFTSYWMHWMRQAPGQGLEWMGTIYPRNSNTDYNQKFKARVTMTRDTSTSTVYMELSSLRSEDTAVYYCARPLYYYVTSPPTLFWGQGTLVTVSS

SEQ ID NO: 140 - protein, 139 aa, Artificial Sequence; identical to SEQ ID NO: 137
  synthetic sequence

SEQ ID NO: 141 - protein, 125 aa, Artificial Sequence
  synthetic sequence
MRAWIFFLLCLAGRALADIQLTQSPSFLSASVGDRVTITCKASQDVGTAVAWYQQKPGKAPKLLIYWASTRHTGVPSRFSGSGSGTEFTLTISSLQPEDFATYYCQQCNSYPFTFGSGTKLEIKR

SEQ ID NO: 142 - protein, 125 aa, Artificial Sequence
  synthetic sequence
MRAWIFFLLCLAGRALADIQLTQSPSFLSASVGDRVTITCKASQDVGTAVAWYQQKPGKAPKLLIYWASTRHTGVPSRFSGSGSGTEFTLTISSLQPEDFATYYCQQAQSYPFTFGSGTKLEIKR

SEQ ID NO: 143 - protein, 125 aa, Artificial Sequence
  synthetic sequence
MRAWIFFLLCLAGRALADIQLTQSPSFLSASVGDRVTITCKASQDVGTAVAWYQQKPGKAPKLLIYWASTRHTGVPSRFSGSGSGTEFTLTISSLQPEDFATYYCQQANSYPFTFGSGTKLEIKR

SEQ ID NO: 144 - protein, 125 aa, Artificial Sequence; identical to SEQ ID NO: 143
  synthetic sequence

SEQ ID NO: 145 - protein, 125 aa, Artificial Sequence
  synthetic sequence
MRAWIFFLLCLAGRALADIQLTQSPSFLSASVGDRVTITCKASQDVGTAVAWYQQKPGKAPKLLIYWASTRHTGVPSRFSGSGSGTEFTLTISSLQPEDFATYYCQQVNSYPFTFGSGTKLEIKR

SEQ ID NO: 146 - protein, 125 aa, Artificial Sequence
  synthetic sequence
MRAWIFFLLCLAGRALADIQLTQSPSFLSASVGDRVTITCKASQDVGTAVAWYQQKPGKAPKLLIYWASTRHTGVPSRFSGSGSGTEFTLTISSLQPEDFATYYCQQINSYPFTFGSGTKLEIKR

SEQ ID NO: 147 - protein, 125 aa, Artificial Sequence
  synthetic sequence
MRAWIFFLLCLAGRALADIQLTQSPSFLSASVGDRVTITCKASQDVGTAVAWYQQKPGKAPKLLIYWASTRHTGVPSRFSGSGSGTEFTLTISSLQPEDFATYYCQQLNSYPFTFGSGTKLEIKR

SEQ ID NO: 148 - protein, 125 aa, Artificial Sequence; identical to SEQ ID NO: 143
  synthetic sequence

SEQ ID NO: 149 - protein, 125 aa, Artificial Sequence
  synthetic sequence
MRAWIFFLLCLAGRALADIQLTQSPSFLSASVGDRVTITCKASQDVGTAVAWYQQKPGKAPKLLIYWASTRHTGVPSRFSGSGSGTEFTLTISSLQPEDFATYYCQQSNSYPFTFGSGTKLEIKR

SEQ ID NO: 150 - protein, 125 aa, Artificial Sequence
  synthetic sequence
MRAWIFFLLCLAGRALADIQLTQSPSFLSASVGDRVTITCKASQDVGTAVAWYQQKPGKAPKLLIYWASTRHTGVPSRFSGSGSGTEFTLTISSLQPEDFATYYCQQASTYPFTFGSGTKLEIKR

SEQ ID NO: 151 - protein, 125 aa, Artificial Sequence
  synthetic sequence
MRAWIFFLLCLAGRALADIQLTQSPSFLSASVGDRVTITCKASQDVGTAVAWYQQKPGKAPKLLIYWASTRHTGVPSRFSGSGSGTEFTLTISSLQPEDFATYYCQQASAYPFTFGSGTKLEIKR

SEQ ID NO: 152 - protein, 125 aa, Artificial Sequence
  synthetic sequence
MRAWIFFLLCLAGRALADIQLTQSPSFLSASVGDRVTITCKASQDVGTAVAWYQQKPGKAPKLLIYWASTRHTGVPSRFSGSGSGTEFTLTISSLQPEDFATYYCQQARTYPFTFGSGTKLEIKR

SEQ ID NO: 153 - protein, 125 aa, Artificial Sequence
  synthetic sequence
MRAWIFFLLCLAGRALADIQLTQSPSFLSASVGDRVTITCKASQDVGTAVAWYQQKPGKAPKLLIYWASTRHTGVPSRFSGSGSGTEFTLTISSLQPEDFATYYCQQARAYPFTFGSGTKLEIKR

SEQ ID NO: 154 - protein, 125 aa, Artificial Sequence
  synthetic sequence
MRAWIFFLLCLAGRALADIQLTQSPSFLSASVGDRVTITCKASQDVGTAVAWYQQKPGKAPKLLIYWASTRHTGVPSRFSGSGSGTEFTLTISSLQPEDFATYYCQQAKTYPFTFGSGTKLEIKR

SEQ ID NO: 155 - protein, 125 aa, Artificial Sequence
  synthetic sequence
MRAWIFFLLCLAGRALADIQLTQSPSFLSASVGDRVTITCKASQDVGTAVAWYQQKPGKAPKLLIYWASTRHTGVPSRFSGSGSGTEFTLTISSLQPEDFATYYCQQAKAYPFTFGSGTKLEIKR

SEQ ID NO: 156 - protein, 125 aa, Artificial Sequence
  synthetic sequence
MRAWIFFLLCLAGRALADIQLTQSPSFLSASVGDRVTITCKASQDVGTAVAWYQQKPGKAPKLLIYWASTRHTGVPSRFSGSGSGTEFTLTISSLQPEDFATYYCQQAQTYPFTFGSGTKLEIKR

SEQ ID NO: 157 - protein, 125 aa, Artificial Sequence
  synthetic sequence
MRAWIFFLLCLAGRALADIQLTQSPSFLSASVGDRVTITCKASQDVGTAVAWYQQKPGKAPKLLIYWASTRHTGVPSRFSGSGSGTEFTLTISSLQPEDFATYYCQQAQAYPFTFGSGTKLEIKR

SEQ ID NO: 158 - protein, 125 aa, Artificial Sequence
  synthetic sequence
MRAWIFFLLCLAGRALADIQLTQSPSFLSASVGDRVTITCKASQDVGTAVAWYQQKPGKAPKLLIYWASTRHTGVPSRFSGSGSGTEFTLTISSLQPEDFATYYCQQYSSYPFTFGSGTKLEIKR

SEQ ID NO: 159 - protein, 125 aa, Artificial Sequence
  synthetic sequence
MRAWIFFLLCLAGRALADIQLTQSPSFLSASVGDRVTITCKASQDVGTAVAWYQQKPGKAPKLLIYWASTRHTGVPSRFSGSGSGTEFTLTISSLQPEDFATYYCQQANTYPFTFGSGTKLEIKR

SEQ ID NO: 160 - protein, 125 aa, Artificial Sequence
  synthetic sequence
MRAWIFFLLCLAGRALADIQLTQSPSFLSASVGDRVTITCKASQDVGTAVAWYQQKPGKAPKLLIYWAFTRHTGVPSRFSGSGSGTEFTLTISSLQPEDFATYYCQQAQSYPFTFGSGTKLEIKR

SEQ ID NO: 161 - protein, 125 aa, Artificial Sequence
  synthetic sequence
MRAWIFFLLCLAGRALADIQLTQSPSFLSASVGDRVTITCKASQDVGTAVAWYQQKPGKAPKLLIYWAATRHTGVPSRFSGSGSGTEFTLTISSLQPEDFATYYCQQAQSYPFTFGSGTKLEIKR

SEQ ID NO: 162 - protein, 125 aa, Artificial Sequence
  synthetic sequence
MRAWIFFLLCLAGRALADIQLTQSPSFLSASVGDRVTITCKASQDVGTAVAWYQQKPGKAPKLLIYWAPTRHTGVPSRFSGSGSGTEFTLTISSLQPEDFATYYCQQAQSYPFTFGSGTKLEIKR

SEQ ID NO: 163 - protein, 125 aa, Artificial Sequence
  synthetic sequence
MRAWIFFLLCLAGRALADIQLTQSPSFLSASVGDRVTITCKASQDVGTAVAWYQQKPGKAPKLLIYWATTRHTGVPSRFSGSGSGTEFTLTISSLQPEDFATYYCQQAQSYPFTFGSGTKLEIKR

SEQ ID NO: 164 - protein, 125 aa, Artificial Sequence
  synthetic sequence
MRAWIFFLLCLAGRALADIQLTQSPSFLSASVGDRVTITCKASQDVGTAVAWYQQKPGKAPKLLIYWAHTRHTGVPSRFSGSGSGTEFTLTISSLQPEDFATYYCQQAQSYPFTFGSGTKLEIKR

SEQ ID NO: 165 - protein, 125 aa, Artificial Sequence
  synthetic sequence
MRAWIFFLLCLAGRALADIQLTQSPSFLSASVGDRVTITCKASQDVGTAVAWYQQKPGKAPKLLIYWANTRHTGVPSRFSGSGSGTEFTLTISSLQPEDFATYYCQQAQSYPFTFGSGTKLEIKR

SEQ ID NO: 166 - protein, 125 aa, Artificial Sequence
  synthetic sequence
MRAWIFFLLCLAGRALADIQLTQSPSFLSASVGDRVTITCKASQDVGTAVAWYQQKPGKAPKLLIYWAYTRHTGVPSRFSGSGSGTEFTLTISSLQPEDFATYYCQQAQSYPFTFGSGTKLEIKR

SEQ ID NO: 167 - protein, 125 aa, Artificial Sequence
  synthetic sequence
MRAWIFFLLCLAGRALADIQLTQSPSFLSASVGDRVTITCKASQDVRTAVAWYQQKPGKAPKLLIYWASTRHTGVPSRFSGSGSGTEFTLTISSLQPEDFATYYCQQAQSYPFTFGSGTKLEIKR

SEQ ID NO: 168 - protein, 125 aa, Artificial Sequence
  synthetic sequence
MRAWIFFLLCLAGRALADIQLTQSPSFLSASVGDRVTITCKASQDVGTAVAWYQQKPGKAPKLLIYVASTRHTGVPSRFSGSGSGTEFTLTISSLQPEDFATYYCQQAQSYPFTFGSGTKLEIKR

SEQ ID NO: 169 - protein, 125 aa, Artificial Sequence
  synthetic sequence
MRAWIFFLLCLAGRALADIQLTQSPSFLSASVGDRVTITCRASQGIRSYLAWYQQKPGKAPKLLIYVASTLQSGVPSRFSGSGSGTEFTLTISSLQPEDFATYYCQQCNSYPFTFGSGTKLEIKR

SEQ ID NO: 170 - protein, 125 aa, Artificial Sequence
  synthetic sequence
MRAWIFFLLCLAGRALADIQLTQSPSFLSASVGDRVTITCRASQGIRSYLAWYQQKPGKAPKLLIYVASTLQSGVPSRFSGSGSGTEFTLTISSLQPEDFATYYCQQCNNYPFTFGSGTKLEIKR

SEQ ID NO: 171 - protein, 125 aa, Artificial Sequence; identical to SEQ ID NO: 154
  synthetic sequence

SEQ ID NO: 172 - protein, 125 aa, Artificial Sequence
  synthetic sequence
MRAWIFFLLCLAGRALADIQLTQSPSFLSASVGDRVTITCRASQGIRSYLAWYQQKPGKAPKLLIYVASTLQSGVPSRFSGSGSGTEFTLTISSLQPEDFATYYCQQANNYPFTFGSGTKLEIKR

SEQ ID NO: 173 - protein, 125 aa, Artificial Sequence
  synthetic sequence
MRAWIFFLLCLAGRALADIQLTQSPSFLSASVGDRVTITCRASQGIRSYLAWYQQKPGKAPKLLIYVASTLQSGVPSRFSGSGSGTEFTLTISSLQPEDFATYYCQQAQSYPFTFGSGTKLEIKR

SEQ ID NO: 174 - protein, 125 aa, Artificial Sequence
  synthetic sequence
MRAWIFFLLCLAGRALADIQLTQSPSFLSASVGDRVTITCRASQGIRSYLAWYQQKPGKAPKLLIYVAFTLQSGVPSRFSGSGSGTEFTLTISSLQPEDFATYYCQQCNSYPFTFGSGTKLEIKR

SEQ ID NO: 175 - protein, 125 aa, Artificial Sequence
  synthetic sequence
MRAWIFFLLCLAGRALADIQLTQSPSFLSASVGDRVTITCRASQGIRSYLAWYQQKPGKAPKLLIYVAFTLQSGVPSRFSGSGSGTEFTLTISSLQPEDFATYYCQQCNNYPFTFGSGTKLEIKR

SEQ ID NO: 176 - protein, 139 aa, Artificial Sequence
  synthetic sequence
MRAWIFFLLCLAGRALAQVQLVQSGAEVKKPGASVKVSCKASGYTFTSYYMHWVRQAPGQGLEWMGTIYPRNSDTDYNQKFKARVTMTRDTSTSTVYMELSSLRSEDTAVYYCARPLYYYVTSPPTLLWGQGTLVTVSS

SEQ ID NO: 177 - protein, 139 aa, Artificial Sequence
  synthetic sequence
MRAWIFFLLCLAGRALAQVQLVQSGAEVKKPGASVKVSCKASGYTFTSYWMHWVRQAPGQGLEWMGIINPSGGSTSYAQKFQGRVTMTRDTSTSTVYMELSSLRSEDTAVYYCARPLYYYVTSPPTLLWGQGTLVTVSS

SEQ ID NO: 178 - protein, 144 aa, Artificial Sequence
  synthetic sequence
MRAWIFFLLCLAGRALAEVQLVESGGGLVQPGGSLRLSCAASGFTFSNYWMNWVRQAPGKGLEWVADIRLKSNNYATHYAESVKGRFTISRDNAKNSLYLQMNSLRAEDTAVYYCVRDGPYWGQGTLVTVSAASTKGPSVFPLA

SEQ ID NO: 179 - protein, 144 aa, Artificial Sequence
  synthetic sequence
MRAWIFFLLCLAGRALAEVQLVESGGGLVQPGGSLRLSCAASGFTFSNYWMNWVRQAPEKGLEWVADIRLKSNNYATHYAESVKGRFTISRDDAKSSLYLQMNNLRAEDTAVYYCVRDGPYWGQGTLVTVSAASTKGPSVFPLA

SEQ ID NO: 180 - protein, 144 aa, Artificial Sequence
  synthetic sequence
MRAWIFFLLCLAGRALAEVQLVESGGGLVQPGGSLRLSCVASGFTFSNYWMNWVRQAPGKGLEWVADIRLKSNNYATHYAESVKGRFTISRDDAKNSVYLQMNSLRAEDTAVYYCVRDGPYWGQGTLVTVSAASTKGPSVFPLA

SEQ ID NO: 181 - protein, 144 aa, Artificial Sequence
  synthetic sequence
MRAWIFFLLCLAGRALAEVQLVESGGGLVQPGGSLRLSCVASGFTFSNYWMNWVRQAPGKGLEWVADIRLKSNNYATHYAESVKGRFTISRDDAKSSVYLQMNSLRAEDTAVYYCVRDGPYWGQGTLVTVSAASTKGPSVFPLA

SEQ ID NO: 182 - protein, 144 aa, Artificial Sequence
  synthetic sequence
MRAWIFFLLCLAGRALAEVQLVESGGGLVQPGGSLRLSCVASGFTFSNYWMNWVRQAPGKGLEWVADIRLKSNNYATHYAESVKGRFTISRDDAKNSVYLQMNSLRAEDTAVYYCVRDAPYWGQGTLVTVSAASTKGPSVFPLA

SEQ ID NO: 183 - protein, 144 aa, Artificial Sequence
  synthetic sequence
MRAWIFFLLCLAGRALAEVQLVESGGGLVQPGGSLRLSCVASGFTFSNYWMNWVRQAPGKGLEWVADIRLKSNNYATHYAESVKGRFTISRDDAKNSVYLQMNSLRAEDTAVYYCVRDSPYWGQGTLVTVSAASTKGPSVFPLA

SEQ ID NO: 184 - protein, 144 aa, Artificial Sequence
  synthetic sequence
MRAWIFFLLCLAGRALAEVQLVESGGGLVQPGGSLRLSCVASGFTFSNYWMNWVRQAPGKGLEWVADIRLKSNNYATHYAESVKGRFTISRDDAKNSVYLQMNSLRAEDTAVYYCVRGGPYWGQGTLVTVSAASTKGPSVFPLA

SEQ ID NO: 185 - protein, 144 aa, Artificial Sequence
  synthetic sequence
MRAWIFFLLCLAGRALAEVQLVESGGGLVQPGGSLRLSCVASGFTFSNYWMNWVRQAPGKGLEWVADIRLKSNNYATHYAESVKGRFTISRDDAKNSVYLQMNSLRAEDTAVYYCVRSGPYWGQGTLVTVSAASTKGPSVFPLA

SEQ ID NO: 186 - protein, 144 aa, Artificial Sequence
  synthetic sequence
MRAWIFFLLCLAGRALAEVQLVESGGGLVQPGGSLRLSCVASGFTFSNYWMNWVRQAPGKGLEWVADIRLKSNNYATHYAESVKGRFTISRDDAKNSVYLQMNSLRAEDTAVYYCVRTGPYWGQGTLVTVSAASTKGPSVFPLA

SEQ ID NO: 187 - protein, 144 aa, Artificial Sequence
  synthetic sequence
MRAWIFFLLCLAGRALAEVQLVESGGGLVQPGGSLRLSCVASGFTFSNYWMNWVRQAPGKGLEWVADIRLKSNNYATHYAESVKGRFTISRDDAKNSVYLQMNSLRAEDTAVYYCVRMGPYWGQGTLVTVSAASTKGPSVFPLA

SEQ ID NO: 188 - protein, 125 aa, Artificial Sequence; identical to SEQ ID NO: 2
  synthetic sequence

SEQ ID NO: 189 - protein, 113 aa, Artificial Sequence; identical to SEQ ID NO: 4
  synthetic sequence

SEQ ID NO: 190 - protein, 130 aa, Artificial Sequence
  synthetic sequence
MRAWIFFLLCLAGRALADVVMTQSPLSLPVTLGQPASISCRSSQSLVYSDGNTYLNWFQQRPGQSPRRLIYKVSNRDSGVPDRFSGSGSGTDFTLKISRVEAEDVGVYYCFQGSHVPRTFGGGTKLEIKR

SEQ ID NO: 191 - protein, 130 aa, Artificial Sequence
  synthetic sequence
MRAWIFFLLCLAGRALADVVMTQSPLSLPVTLGQPASISCRSSQSIVHSNGNTYLEWYLQRPGQSPRLLIYKISNRFSGVPDRFSGSGSGTDFTLKISRVEAEDVGVYYCFQGSHVPRTFGGGTKLEIKR

SEQ ID NO: 192 - protein, 130 aa, Artificial Sequence
  synthetic sequence
MRAWIFFLLCLAGRALADVLMTQSPLSLPVTLGQPASISCRSSQSIVHSNGNTYLEWYQQRPGQSPRLLIYKISNRFSGVPDRFSGSGSGTDFTLKISRVEAEDVGVYYCFQGSHVPRTFGGGTKLEIKR

SEQ ID NO: 193 - protein, 130 aa, Artificial Sequence
  synthetic sequence
MRAWIFFLLCLAGRALADVVMTQSPLSLPVTLGQPASISCRSSQSIVHSNGNTYLEWYQQRPGQSPRLLIYKISNRFSGVPDRFSGSGSGTDFTLKISRVEAEDVGVYYCFQGSHVPRTFGGGTKLEIKR

SEQ ID NO: 194 - protein, 130 aa, Artificial Sequence
  synthetic sequence
MRAWIFFLLCLAGRALADVVMTQSPLSLPVTLGQPASISCRSSQSIVHSQGNTYLEWYQQRPGQSPRLLIYKISNRFSGVPDRFSGSGSGTDFTLKISRVEAEDVGVYYCFQGSHVPRTFGGGTKLEIKR

SEQ ID NO: 195 - protein, 130 aa, Artificial Sequence
  synthetic sequence
MRAWIFFLLCLAGRALADVVMTQSPLSLPVTLGQPASISCRSSQSIVHSLGNTYLEWYQQRPGQSPRLLIYKISNRFSGVPDRFSGSGSGTDFTLKISRVEAEDVGVYYCFQGSHVPRTFGGGTKLEIKR

SEQ ID NO: 196 - protein, 130 aa, Artificial Sequence
  synthetic sequence
MRAWIFFLLCLAGRALADVVMTQSPLSLPVTLGQPASISCRSSQSIVHSAGNTYLEWYQQRPGQSPRLLIYKISNRFSGVPDRFSGSGSGTDFTLKISRVEAEDVGVYYCFQGSHVPRTFGGGTKLEIKR

SEQ ID NO: 197 - protein, 130 aa, Artificial Sequence
  synthetic sequence
MRAWIFFLLCLAGRALADVVMTQSPLSLPVTLGQPASISCRSSQSIVHSSGNTYLEWYQQRPGQSPRLLIYKISNRFSGVPDRFSGSGSGTDFTLKISRVEAEDVGVYYCFQGSHVPRTFGGGTKLEIKR

SEQ ID NO: 198 - protein, 130 aa, Artificial Sequence
  synthetic sequence
MRAWIFFLLCLAGRALADVVMTQSPLSLPVTLGQPASISCRSSQSIVHSEGNTYLEWYQQRPGQSPRLLIYKISNRFSGVPDRFSGSGSGTDFTLKISRVEAEDVGVYYCFQGSHVPRTFGGGTKLEIKR

SEQ ID NO: 199 - protein, 130 aa, Artificial Sequence
  synthetic sequence
MRAWIFFLLCLAGRALADVVMTQSPLSLPVTLGQPASISCRSSQSIVHSTGNTYLEWYQQRPGQSPRLLIYKISNRFSGVPDRFSGSGSGTDFTLKISRVEAEDVGVYYCFQGSHVPRTFGGGTKLEIKR

SEQ ID NO: 200 - protein, 130 aa, Artificial Sequence
  synthetic sequence
MRAWIFFLLCLAGRALADVVMTQSPLSLPVTLGQPASISCRSSQSIVHSNANTYLEWYQQRPGQSPRLLIYKISNRFSGVPDRFSGSGSGTDFTLKISRVEAEDVGVYYCFQGSHVPRTFGGGTKLEIKR

SEQ ID NO: 201 - protein, 130 aa, Artificial Sequence
  synthetic sequence
MRAWIFFLLCLAGRALADVVMTQSPLSLPVTLGQPASISCRSSQSIVHSNDNTYLEWYQQRPGQSPRLLIYKISNRFSGVPDRFSGSGSGTDFTLKISRVEAEDVGVYYCFQGSHVPRTFGGGTKLEIKR

SEQ ID NO: 202 - protein, 130 aa, Artificial Sequence
  synthetic sequence
MRAWIFFLLCLAGRALADVVMTQSPLSLPVTLGQPASISCRSSQSIVHSFGNTYLEWYQQRPGQSPRLLIYKISNRFSGVPDRFSGSGSGTDFTLKISRVEAEDVGVYYCFQGSHVPRTFGGGTKLEIKR

SEQ ID NO: 203 - protein, 130 aa, Artificial Sequence
  synthetic sequence
MRAWIFFLLCLAGRALADVVMTQSPLSLPVTLGQPASISCRSSQSIVHSNVNTYLEWYQQRPGQSPRLLIYKISNRFSGVPDRFSGSGSGTDFTLKISRVEAEDVGVYYCFQGSHVPRTFGGGTKLEIKR

SEQ ID NO: 204 - protein, 130 aa, Artificial Sequence; identical to SEQ ID NO: 192
  synthetic sequence

SEQ ID NO: 205 - protein, 130 aa, Artificial Sequence; identical to SEQ ID NO: 193
  synthetic sequence

SEQ ID NO: 206 - protein, 153 aa, Artificial Sequence
  synthetic sequence
MEFGLSWVFLVALLRGVQCQVQLVESGGGVVQPGRSLRLSCAASGFTFSSYAMHWVRQAPGKGLEWVAVISYDGSNKYYADSVKGRFTISRDNSKNTLYLQMNSLRADDTAVYYCAKAPGPMVRGVIPDIWGQGTMVTVSSGSASAPTLFPLA

SEQ ID NO: 207 - protein, 153 aa, Artificial Sequence
  synthetic sequence
MEFGLSWVFLVALLRGVQCQVQLVESGGGVVQPGRSLRLSCAASGFTFSNYAMHWVRQAPGKGLEWVAVISYDGNNKYYADSVKGRFTISRDNFKNTLYLQMNSLRADDTAVYYCAKAPGSMVRGVIPDIWGQGTMVTVSSGSASAPTLFPLA

SEQ ID NO: 208 - protein, 153 aa, Artificial Sequence
  synthetic sequence
MEFGLSWVFLVALLRGVQCQVQLVESGGGVVQPGRSLRLSCAASGFTFSSYVMHWVRQAPGKGLEWVAVISYDGSNKYYADSVKGRFTISRDNSNNTLYLQMNSLRADDTAVYYCAKAPGPMVRGVIPDIWGQGTMVTVSSGSASAPTLFPLA

SEQ ID NO: 209 - protein, 153 aa, Artificial Sequence
  synthetic sequence
MEFGLSWVFLVALLRGVQCQVQLVESGGGVVQPGRSLRLSCAASGFTFSNYVMHWVRQTPGKGLEWVAVISYDGRNKYYADSVKGRFTISRDNFNNTLYLQMNSLRADDTAVYYCAKAPGPMVRGVIPDIWGQGTMVTVSSGSASAPTLFPLA

SEQ ID NO: 210 - protein, 153 aa, Artificial Sequence
  synthetic sequence
MEFGLSWVFLVALLRGVQCQVQLVESGGGVVQPGRSLRLSCAASGFTFSNYVMHWVRQTPGKGLEWVAVISYDGRNKYYADSVKGRFTISRDNFNNTLYLQMNSLRADDTAVYYCAKAPGSMVRGVIPDIWGQGTMVTVSSGSASAPTLFPLA

SEQ ID NO: 211 - protein, 153 aa, Artificial Sequence
  synthetic sequence
MEFGLSWVFLVALLRGVQCQVQLVESGGGVVQPGRSLRLSCAASGFTFSNYVMHWVRQTPGKGLEWVAVISYDGRNKYYADSVKGRFTISRDNSNNTLYLQMNSLRAEDTAVYYCAKAPGPMVRGVIPDIWGQGTMVTVSSGSASAPTLFPLA

SEQ ID NO: 212 - protein, 153 aa, Artificial Sequence
  synthetic sequence
MEFGLSWVFLVALLRGVQCQVQLVESGGGVVQPGRSLRLSCAASGFTFSNYVMHWVRQTPGKGLEWVAVISYDGRNKYYADSVKGRFTISRDNSNNTLYLQMNSLRADDTAVYYCAKAPGSMVRGVIPDIWGQGTMVTVSSGSASAPTLFPLA

SEQ ID NO: 213 - protein, 153 aa, Artificial Sequence
  synthetic sequence
MEFGLSWVFLVALLRGVQCQVQLVESGGGVVQPGRSLRLSCAASGFTFSNYVMHWVRQTPGKGLEWVAVISYDGRNKYYADSVKGRFTISRDNFKNTLYLQMNSLRADDTAVYYCAKAPGSMVRGVIPDIWGQGTMVTVSSGSASAPTLFPLA

SEQ ID NO: 214 - protein, 153 aa, Artificial Sequence
  synthetic sequence
MEFGLSWVFLVALLRGVQCQVQLVESGGGVVQPGRSLRLSCAASGFTFSNYVMHWVRQAPGKGLEWVAVISYDGSNKYYADSVKGRFTISRDNSNNTLYLQMNSLRADDTAVYYCAKAPGPMVRGVIPDIWGQGTMVTVSSGSASAPTLFPLA

SEQ ID NO: 215 - protein, 153 aa, Artificial Sequence; identical to SEQ ID NO: 214
  synthetic sequence

SEQ ID NO: 216 - protein, 153 aa, Artificial Sequence
  synthetic sequence
MEFGLSWVFLVALLRGVQCQVQLVESGGGVVQPGRSLRLSCAASGFTFSNYVMHWVRQTPGKGLEWVAVISYDGRNKYYADSVKGRFTISRDNSNNTLYLQMNSLRADDTAVYYCAKAPGPMVRGVIPDIWGQGTMVTVSSGSASAPTLFPLA

SEQ ID NO: 217 - protein, 153 aa, Artificial Sequence; identical to SEQ ID NO: 216
  synthetic sequence

SEQ ID NO: 218 - protein, 130 aa, Artificial Sequence
  synthetic sequence
METPAQLLFLLLLWLPDTTGEIVLTQSPGTLSLSPGERATLSCRASQSVSSSYLAWYQQKPGQAPRLLIYGASSRATGIPDRFSGSGSGTDFTLTISRLEPEDFAVYYCMQGTHWPPYTFGQGTKLEIKR

SEQ ID NO: 219 - protein, 130 aa, Artificial Sequence
  synthetic sequence
METPAQLLFLLLLWLPDTTGEIVLTQSPGTLSLSPGERATLSCRASQSVISSYLAWYQQKPGQAPRLLIYGTSSRATGIPDRFSGSGSGTDFTLTISRLEPEDFAVYYCMQGTHWLPYTFGQGTKLEIKR

SEQ ID NO: 220 - protein, 130 aa, Artificial Sequence
  synthetic sequence
METPAQLLFLLLLWLPDTTGEIVLTQSPGTLSLSPGERATLSCRTSQSVRSSYLGWYQQKPGQVPRLLIYGASSRATGIPDRFSGSGSGTDFTLTISRLEPEDFAVYYCMQATHWPPYTFGQGTKLEIKR

SEQ ID NO: 221 - protein, 130 aa, Artificial Sequence
  synthetic sequence
METPAQLLFLLLLWLPDTTGEIVLTQSPGTLSLSPGERATLSCRTSQSVRSSYLGWYQQKPGQVPRLLIYGTSIRATGIPDRFSGSGSGTDFTLTISRLEPEDFAVYYCMQATHWPPYTFGQGTKLEIKR

SEQ ID NO: 222 - protein, 147 aa, Artificial Sequence
  synthetic sequence
METPAQLLFLLLLWLPDTTGEIVLTQSPGTLSLSPGERATLSCRTSQSVRSSYLGWYQQKPGQVPRLLIYGTSIRATGIPDRFSGSGSGTDFTLTISRLEPEDFAVYYCMQATHWPPYTFGQGTKLEIKRTVAAPSVFIFPPSDEQL

SEQ ID NO: 223 - protein, 130 aa, Artificial Sequence
  synthetic sequence
METPAQLLFLLLLWLPDTTGEIVLTQSPGTLSLSPGERATLSCRASQSVISSYLAWYQQKPGQTPRLLIYGTSSRATGIPDRFSGSGSGTDFTLTISRLEPEDFAVYYCMQGTHWLPYTFGQGTKLEIKR

SEQ ID NO: 224 - protein, 130 aa, Artificial Sequence
  synthetic sequence
METPAQLLFLLLLWLPDTTGEIVLTQSPGTLSLSPGERATLSCRASQSVISSYLAWYQQKPGQAPRLLIYGTSIRATGIPDRFSGSGSGTDFTLTISRLEPEDFAVYYCMQGTHWLPYTFGQGTKLEIKR

SEQ ID NO: 225 - protein, 130 aa, Artificial Sequence
  synthetic sequence
METPAQLLFLLLLWLPDTTGEIVLTQSPGTLSLSPGERATLSCRTSQSVRSSYLAWYQQKPGQAPRLLIYGASSRATGIPDRFSGSGSGTDFTLTISRLEPEDFAVYYCMQATHWPPYTFGQGTKLEIKR

SEQ ID NO: 226 - protein, 130 aa, Artificial Sequence
  synthetic sequence
METPAQLLFLLLLWLPDTTGEIVLTQSPGTLSLSPGERATLSCRTSQSVRSSYLGWYQQKPGQAPRLLIYGASSRATGIPDRFSGSGSGTDFTLTISRLEPEDFAVYYCMQATHWPPYTFGQGTKLEIKR

SEQ ID NO: 227 - protein, 130 aa, Artificial Sequence
  synthetic sequence
METPAQLLFLLLLWLPDTTGEIVLTQSPGTLSLSPGERATLSCRTSQSVSSSYLGWYQQKPGQAPRLLIYGASSRATGIPDRFSGSGSGTDFTLTISRLEPEDFAVYYCMQATHWPPYTFGQGTKLEIKR

SEQ ID NO: 228 - protein, 130 aa, Artificial Sequence
  synthetic sequence
METPAQLLFLLLLWLPDTTGEIVLTQSPGTLSLSPGERATLSCRTSQSVSSSYLGWYQQKPGQVPRLLIYGASSRATGIPDRFSGSGSGTDFTLTISRLEPEDFAVYYCMQATHWPPYTFGQGTKLEIKR

SEQ ID NO: 229 - protein, 130 aa, Artificial Sequence
  synthetic sequence
METPAQLLFLLLLWLPDTTGEIVLTQSPGTLSLSPGERATLSCRTSQSVSSSYLAWYQQKPGQVPRLLIYGASSRATGIPDRFSGSGSGTDFTLTISRLEPEDFAVYYCMQATHWPPYTFGQGTKLEIKR

SEQ ID NO: 230 - protein, 130 aa, Artificial Sequence
  synthetic sequence
METPAQLLFLLLLWLPDTTGEIVLTQSPGTLSLSPGERATLSCRTSQSVSSSYLAWYQQKPGQAPRLLIYGASSRATGIPDRFSGSGSGTDFTLTISRLEPEDFAVYYCMQATHWPPYTFGQGTKLEIKR

SEQ ID NO: 231 - protein, 130 aa, Artificial Sequence
  synthetic sequence
METPAQLLFLLLLWLPDTTGEIVLTQSPGTLSLSPGERATLSCRASQSVSSSYLAWYQQKPGQAPRLLIYGASSRATGIPDRFSGSGSGTDFTLTISRLEPEDFAVYYCMQATHWPPYTFGQGTKLEIKR

(19) United States
(12) Patent Application Publication
(10) Pub. No.: US 2019/0016795 A1
(43) Pub. Date: Jan. 17, 2019

(54) INHIBITION OF ALLERGIC REACTION USING AN IL-33 INHIBITOR

(71) Applicant: AnaptysBio, Inc., San Diego, CA (US)

(72) Inventor: Marco LONDEI, San Diego, CA (US)

(73) Assignee: AnaptysBio, Inc., San Diego, CA (US)

(21) Appl. No.: 16/070,200

(22) PCT Filed: Jan. 17, 2017
(86) PCT No.: PCT/US17/13818

§ 371 Date: Jul. 13, 2018

Related U.S. Application Data
(60) Provisional application No. 62/278,671, filed on Jan. 14, 2016.

(51) Int. Cl. C07K 16/24

(52) U.S. Cl. CPC C07K 16/244; C07K 2317/56; C07K 2317/565; A61K 2039/505; C07K 2317/92; C07K 2317/76; C07K 2317/94

(57) ABSTRACT

A method of inhibiting or preventing an allergic reaction, particularly to a food antigen, in a mammal comprising administering to the mammal an IL-33 inhibitor.

### PRIORITY INFORMATION

This application claims the benefit of U.S. Provisional Application No. 62/278,671, filed Jan. 14, 2016. The disclosure of this provisional application is incorporated herein in its entirety for all purposes.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 283,263 Byte ASCII (Text) file named “727217_ST25.txt,” created on Jan. 13, 2017.

### BACKGROUND OF THE INVENTION

A significant proportion of individuals suffer from food allergies, which represent an increasing socio-economic burden. Many food antigens can trigger an allergic reaction which, in the most severe cases, might lead to anaphylactic shock and death. The most common cause of food allergy is peanut allergy, which afflicts up to 10% of children and 1% of adults in the US. Once an allergic reaction occurs patients might manifest with an array of symptoms and in extreme cases they have to be hospitalized and rapidly treated.

As for all atopic disorders, high levels of immunoglobulin E (IgE), and peanut protein-specific IgE, have been described in peanut allergy patients. Current therapy involves education around the avoidance of peanuts in foods, which can be problematic given the wide and frequently hidden use of peanut as a food ingredient. Furthermore, an avoidance approach does not represent a true “therapy.”

Anaphylaxis presents in a significant proportion of peanut allegic individuals and in the most severe reaction patients might be hospitalized and if not properly and rapidly treated might die. In these cases the prompt recognition and treatment of anaphylactic reactions must happen as soon as they occur. The only effective treatment for anaphylaxis is intramuscular epinephrine (EpiPen) administration, whilst oxygen, nebulized albuterol, systemic corticosteroids and histamine H1 and/or H2 receptor antagonists can help alleviate secondary symptoms.

Immunotherapeutic approaches, mainly based on antigen-specific (i.e., peanut) desensitization, are being explored to find an effective treatment for this life-threatening allergy. Other strategies are based on antigen desensitization, whereby pathogenic antigen specific T cells are re-educated after a chronic and prolonged challenge with the triggering allergen/antigen. Clinical studies have provided intriguing evidence of potential effects on pathogenic T cells with some clinical benefits. However desensitizing therapeutic approaches bear the intrinsic risk of eliciting an anaphylactic shock reaction, and therefore such approaches may be limited to milder patients at lower risk of anaphylactic response to the desensitizing therapy. Furthermore patients with food allergies often are allergic to more than one food antigen and present with other atopic diseases. These desensitization approaches are specific, by definition, for a single antigen, and do not directly target the cause of the anaphylactic shock reaction and in some cases might actually induce a this reaction.

Thus, a need for new methods for inhibiting or preventing allergic reactions remains.

### BRIEF SUMMARY OF THE INVENTION

The invention provides a method for inhibiting or preventing an allergic reaction in a mammal comprising administering an IL-33 inhibitor to the mammal. Related methods and compositions also are provided.

### DETAILED DESCRIPTION OF THE INVENTION

The invention provides a method for inhibiting or preventing an allergic reaction in a mammal comprising administering an IL-33 inhibitor to the mammal, particularly an allergic reaction to a food antigen (i.e., a food allergy). The term “food allergy,” as used herein, refers to a chronic or acute immunological hypersensitivity reaction (e.g. a type I hypersensitivity reaction) elicited in a mammal in response to an ingested material or “food antigen” (e.g. nuts, peanuts, shellfish, fish, milk, eggs, wheat, and soybeans). In this sense, a “food antigen” is used synonymously with a “food allergen.” Identification and diagnosis of food allergy is routine among persons of ordinary skill in the art. Clinical manifestations of a food allergic reaction include, but are not limited to, rash, eczema, atopic dermatitis, hives, urticaria, angiodedma, asthma, rhinitis, wheezing, sneezing, dyspnea, swelling of the airways, shortness of breath, other respiratory symptoms, abdominal pain, cramping, nausea, vomiting, diarrhea, melena, tachycardia, hypotension, syncope, seizures, and anaphylactic shock. Methods of diagnosing food allergy include, for example, elimination diets, oral food challenge with a suspected allergen, skin prick tests, detection of allergen-specific immunoglobulins (e.g. IgE) within the blood of a subject, and molecular allergy component testing. Allergic response may be defined by increased level of serum IgE or increased expression and secretion of IL-9 compared to the levels in a negative control (e.g., serum levels of a normal, non-diseased subject of the same type, known to be in a non-allergic state, or of a given individual before contacted with a particular allergen), and/or clinical manifestation of anaphylactic shock.

Without wishing to be bound by any particular theory or mechanism of action, it is believed that an allergic reaction, particularly to a food allegen, involves IL-33 or an increase in IL-33 activity, causing or worsening the allergic response. Thus, treatment with an IL-33 inhbitor reduces or eliminates the allergic reaction or symptoms thereof.

It is further believed that the methods provided herein can, in at least some embodiments, target different key steps of the pathogenic cascade controlling peanut allergic reactions from commencement, function of allergen specific pathogenic T cells to the containment of anaphylactic manifestations. IL-33 is a preformed cytokine, which is predominantly present in cells with barrier function, such as endothelial and epithelial cells. It is released rapidly upon allergen, pathogen, or environmental agent challenge, thus acting as a primary and initial trigger of Th2 inflammatory responses. IL-33 pleiotropic functions expand to the orchestration of immune responses by affecting pathogenic Th2 T cells to the magnification of anpylatic responses via IL-33 direct activity on mast cells and bashopils. Therefore, it is believed that IL-33 inhibition controls, at different stages, pathogenic allergic peanut (food) allergic response. IL-33 inhibition could also benefit peanut allergic patients who present with concominant food allergies (i.e. nuts), or with other atopic disorders such as atopic dermatitis or asthma, which are often present in peanut (food) allergy patients. Therefore IL-33 inhibition would represent an holistic therapeutic approach for diseases that share this patoghenic cascade further helping peanut (food) allergy patients.

The method provided herein can be used to treat any mammal, particularly a human. It is believed the method is particularly effective on mammals or humans with a genetic mutation that increases IL-33 expression or signaling, especially after exposure to an antigen (e.g., food antigen).

IL-33 (also known as nuclear factor (NF) in high endothelial venules (NF-HEV)) is a cytokine of the IL-1 family, which also includes the inflammatory cytokines IL-1α, IL-1β, and IL-18. IL-33 has been shown to signal via the ST2 receptor and the IL1RAP receptor. IL-33 is expressed broadly in various tissues, including stomach, lung, spinal cord, brain, and skin, as well as in cells, including smooth muscle cells and epithelial cells lining bronchus and small airways. IL-33 expression is induced by IL-1β and tumor necrosis factor-α (TNF-α) in lung and dermal fibroblasts and, to a lesser extent, by macrophage activation. IL-33 treatment has been shown to induce T-helper (Th) type 2 responses in mice as indicated by an increase in Th2 cytokine production and serum immunoglobulin. Systemic treatment of mice with IL-33 results in pathologic changes in the lung and the digestive tract (see, e.g., Choi et al., Blood, 114(14): 3117-3126 (2009); and Yagami et al., J. Immunology, 185(10): 5743-5750 (2010)).

IL-33 is produced as a 30-kDa precursor protein that is cleaved in vitro by caspase-1, releasing the mature 18-kDa form (see, e.g., Schmitz et al., Immunity, 23(5): 479-490(2005)). Upon binding to the ST2 receptor, IL-33 promotes the activation of nuclear factor (NF)-κB and mitogen-activated protein kinase (MAPK), leading to increased transcription of Th2 cytokines (Schmitz et al., supra).

Any IL-33 inhibitor can be used in accordance with the invention. The IL-33 inhibitor can be a molecule that inhibits IL-33 protein expression (e.g., an antisense or siRNA). Alternatively, the IL-33 inhibitor can be a molecule that blocks the binding of IL-33 to receptors ST2 and IL1RAP. For instance, the IL-33 inhibitor can be an isolated or purified epitope of IL-33 which blocks binding of IL-33 to its receptor in an indirect or allosteric manner. The IL-33 inhibitor can be an IL-33 binding agent, which can be any substance capable of binding or interacting with IL-33 and affecting the biological activity thereof. The IL-33-binding agent can bind an epitope of IL-33 which blocks the binding of IL-33 to receptors ST2 (also known as IL1RL1) and/or IL-1 Receptor Accessory Protein (IL1RAP) and inhibits IL-33 mediated signaling. For example, an IL-33 binding agent may comprise an IL-33 receptor or fragment thereof. In one embodiment, the IL-33 binding agent comprises the IL-33 binding domain of ST2. In another embodiment, the IL-33 binding domain of ST2 is fused to a heterologous polypeptide, for example an Fc portion of an immunoglobulin. The IL-33 binding agent also can be an immunoglobulin or antibody antigen-binding antibody fragment thereof, examples of which are described herein. Other inhibitors of IL-33 expression or activity may include, for example, antibodies that block ST2, IL-1RAP, acrolein, artesunate, vitexin, I-Theanine, or vinpocentine.

The term “immunoglobulin” or “antibody,” as used herein, refers to a protein that is found in blood or other bodily fluids of vertebrates, which is used by the immune system to identify and neutralize foreign objects, such as bacteria and viruses. The polypeptide is “isolated” in that it is removed from its natural environment. In a preferred embodiment, an immunoglobulin or antibody is a protein that comprises at least one complementarity determining region (CDR). The CDRs form the “hypervariable region” of an antibody, which is responsible for antigen binding (discussed further below). A whole immunoglobulin typically consists of four polypeptides: two identical copies of a heavy (H) chain polypeptide and two identical copies of a light (L) chain polypeptide. Each of the heavy chains contains one N-terminal variable (V_{H}) region and three C-terminal constant (C_{H}1, C_{H}2, and C_{H}3) regions, and each light chain contains one N-terminal variable (V_{L}) region and one C-terminal constant (C_{L}) region. The light chains of antibodies can be assigned to one of two distinct types, either kappa (κ) or lambda (λ), based upon the amino acid sequences of their constant domains. In a typical immunoglobulin, each light chain is linked to a heavy chain by disulphide bonds, and the two heavy chains are linked to each other by disulphide bonds. The light chain variable region is aligned with the variable region of the heavy chain, and the light chain constant region is aligned with the first constant region of the heavy chain. The remaining constant regions of the heavy chains are aligned with each other.

The variable regions of each pair of light and heavy chains form the antigen binding site of an antibody. The V_{H} and V_{L} regions have the same general structure, with each region comprising four framework (FW or FR) regions. The term “framework region,” as used herein, refers to the relatively conserved amino acid sequences within the variable region which are located between the hypervariable or complementary determining regions (CDRs). There are four framework regions in each variable domain, which are designated FR1, FR2, FR3, and FR4. The framework regions form the β sheets that provide the structural framework of the variable region (see, e.g., C. A. Janeway et al. (eds.), Immunobiology, 5th Ed., Garland Publishing, New York, N.Y. (2001)).

The framework regions are connected by three complementarity determining regions (CDRs). As discussed above, the three CDRs, known as CDR1, CDR2, and CDR3, form the “hypervariable region” of an antibody, which is responsible for antigen binding. The CDRs form loops connecting, and in some cases comprising part of, the beta-sheet structure formed by the framework regions. While the constant regions of the light and heavy chains are not directly involved in binding of the antibody to an antigen, the constant regions can influence the orientation of the variable regions. The constant regions also exhibit various effector functions, such as participation in antibody-dependent complement-mediated lysis or antibody-dependent cellular toxicity via interactions with effector molecules and cells.

Antibodies which bind to IL-33, and components thereof, are known in the art (see, e.g., US 2014/0271658, US 2009/0041718 A1, 2012/0263709 A1, WO2015099175; WO 2016077381; and WO 2016/077366). Anti-IL-33 antibodies also are commercially available from sources such as, for example, Abcam (Cambridge, Mass.). Antibodies to ST2 or ST2L are disclosed, for example, in US 2014/0004107 and U.S. Pat. No. 9,090,694.

The anti-IL-33 antibody can comprise an immunoglobulin heavy chain polypeptide that comprises an amino acid sequence of any one of SEQ ID NO: 1, SEQ ID NO: 2, SEQ ID NOs: 5-50, SEQ ID NOs: 67-140, SEQ ID NO: 176, SEQ ID NO: 177, SEQ ID NOs: 178-188, and SEQ ID NOs: 206-217, or an amino acid sequence that is at least 90% identical to any one of SEQ ID NO: 1, SEQ ID NO: 2, SEQ ID NOs: 5-50, SEQ ID NOs: 67-140, SEQ ID NO: 176, SEQ ID NO: 177, SEQ ID NOs: 178-188, and SEQ ID NOs: 206-217. In one embodiment of the invention, the isolated immunoglobulin heavy chain polypeptide comprises, consists of, or consists essentially of an amino acid sequence of any one of SEQ ID NO: 1, SEQ ID NO: 2, SEQ ID NOs: 5-50, SEQ ID NOs: 67-140, SEQ ID NO: 176, SEQ ID NO: 177, SEQ ID NOs: 178-188, and SEQ ID NOs: 206-217. When the immunoglobulin heavy chain polypeptide consists essentially of an amino acid sequence of any one of SEQ ID NO: 1, SEQ ID NO: 2, SEQ ID NOs: 5-50, SEQ ID NOs: 67-140, SEQ ID NO: 176, SEQ ID NO: 177, SEQ ID NOs: 178-188, and SEQ ID NOs: 206-217, additional components can be included in the polypeptide that do not materially affect the polypeptide (e.g., protein moieties such as biotin that facilitate purification or isolation). When the immunoglobulin heavy chain polypeptide consists of an amino acid sequence of any one of SEQ ID NO: 1, SEQ ID NO: 2, SEQ ID NOs: 5-50, SEQ ID NOs: 67-140, SEQ ID NO: 176, SEQ ID NO: 177, SEQ ID NOs: 178-188, and SEQ ID NOs: 206-217, the polypeptide does not comprise any additional components (i.e., components that are not endogenous to the inventive immunoglobulin heavy chain polypeptide).

The immunoglobulin heavy chain polypeptide can comprise an amino acid sequence that is at least 90% identical (e.g., at least 91%, at least 92%, at least 93%, at least 94%, at least 95%, at least 96%, at least 97%, at least 98%, at least 99%, or 100% identical) to any one of SEQ ID NO: 1, SEQ ID NO: 2, SEQ ID NOs: 5-50, SEQ ID NOs: 67-140, SEQ ID NO: 176, SEQ ID NO: 177, SEQ ID NOs: 178-188, or SEQ ID NOs: 206-217. Nucleic acid or amino acid sequence “identity,” as described herein, can be determined by comparing a nucleic acid or amino acid sequence of interest to a reference nucleic acid or amino acid sequence. The percent identity is the number of nucleotides or amino acid residues that are the same (i.e., that are identical) as between the sequence of interest and the reference sequence divided by the length of the longest sequence (i.e., the length of either the sequence of interest or the reference sequence, whichever is longer). A number of mathematical algorithms for obtaining the optimal alignment and calculating identity between two or more sequences are known and incorporated into a number of available software programs. Examples of such programs include CLUSTAL-W, T-Coffee, and ALIGN (for alignment of nucleic acid and amino acid sequences), BLAST programs (e.g., BLAST 2.1, BL2SEQ, and later versions thereof) and FASTA programs (e.g., FASTA3×, FASTM, and SSEARCH) (for sequence alignment and sequence similarity searches). Sequence alignment algorithms also are disclosed in, for example, Altschul et al., J. Molecular Biol., 215(3): 403-410 (1990), Beigert et al., Proc. Natl. Acad. Sci. USA, 106(10): 3770-3775 (2009), Durbin et al., eds., Biological Sequence Analysis: Probabilistic Models of Proteins and Nucleic Acids, Cambridge University Press, Cambridge, UK (2009), Soding, Bioinformatics, 21(7): 951-960 (2005), Altschul et al., Nucleic Acids Res., 25(17): 3389-3402 (1997), and Gusfield, Algorithms on Strings, Trees and Sequences, Cambridge University Press, Cambridge UK (1997)).

The anti-IL-33 antibody can comprise an immunoglobulin light chain polypeptide that comprises an amino acid sequence of any one of SEQ ID NO: 3, SEQ ID NO: 4, SEQ ID NOs: 51-66, SEQ ID NOs: 141-175, SEQ ID NOs: 189-205, and SEQ ID NOs: 218-231, or an amino acid sequence that is at least 90% identical to any one of SEQ ID NO: 3, SEQ ID NO: 4, SEQ ID NOs: 51-66, SEQ ID NOs: 141-175, SEQ ID NOs: 189-205, and SEQ ID NOs: 218-231. In one embodiment of the invention, the isolated immunoglobulin light chain polypeptide comprises, consists of, or consists essentially of an amino acid sequence of any one of SEQ ID NO: 3, SEQ ID NO: 4, SEQ ID NOs: 51-66, SEQ ID NOs: 141-175, SEQ ID NOs: 189-205, and SEQ ID NOs: 218-231. When the immunoglobulin light chain polypeptide consists essentially of an amino acid sequence of any one of SEQ ID NO: 3, SEQ ID NO: 4, SEQ ID NOs: 51-66, SEQ ID NOs: 141-175, SEQ ID NOs: 189-205, and SEQ ID NOs: 218-231, additional components can be included in the polypeptide that do not materially affect the polypeptide (e.g., protein moieties such as biotin that facilitate purification or isolation). When the immunoglobulin light chain polypeptide consists of an amino acid sequence of any one of SEQ ID NO: 3, SEQ ID NO: 4, SEQ ID NOs: 51-66, SEQ ID NOs: 141-175, SEQ ID NOs: 189-205, and SEQ ID NOs: 218-231, the polypeptide does not comprise any additional components (i.e., components that are not endogenous to the immunoglobulin light chain polypeptide).

The immunoglobulin light chain polypeptide also can comprise an amino acid sequence that is at least 90% identical (e.g., at least 91%, at least 92%, at least 93%, at least 94%, at least 95%, at least 96%, at least 97%, at least 98%, at least 99%, or 100% identical) to any one of SEQ ID NO: 3, SEQ ID NO: 4, SEQ ID NOs: 51-66, SEQ ID NOs: 141-175, SEQ ID NOs: 189-205, or SEQ ID NOs: 218-231. Nucleic acid or amino acid sequence “identity,” as described herein, can be determined using the methods described herein.

One or more amino acids of the aforementioned immunoglobulin heavy chain polypeptides and/or light chain polypeptides can be replaced or substituted with a different amino acid. An amino acid “replacement” or “substitution” refers to the replacement of one amino acid at a given position or residue by another amino acid at the same position or residue within a polypeptide sequence.

Amino acids are broadly grouped as “aromatic” or “aliphatic.” An aromatic amino acid includes an aromatic ring. Examples of “aromatic” amino acids include histidine (H or His), phenylalanine (F or Phe), tyrosine (Y or Tyr), and tryptophan (W or Trp). Non-aromatic amino acids are broadly grouped as “aliphatic.” Examples of “aliphatic” amino acids include glycine (G or Gly), alanine (A or Ala), valine (V or Val), leucine (L or Leu), isoleucine (I or Ile), methionine (M or Met), serine (S or Ser), threonine (T or Thr), cysteine (C or Cys), proline (P or Pro), glutamic acid (E or Glu), aspartic acid (A or Asp), asparagine (N or Asn), glutamine (Q or Gln), lysine (K or Lys), and arginine (R or Arg).

Aliphatic amino acids may be sub-divided into four sub-groups. The “large aliphatic non-polar sub-group” consists of valine, leucine, and isoleucine. The “aliphatic slightly-polar sub-group” consists of methionine, serine, threonine, and cysteine. The “aliphatic polar/charged sub-group” consists of glutamic acid, aspartic acid, asparagine, glutamine, lysine, and arginine. The “small-residue sub-group” consists of glycine and alanine. The group of charged/polar amino acids may be sub-divided into three sub-groups: the “positively-charged sub-group” consisting of lysine and arginine, the “negatively-charged sub-group” consisting of glutamic acid and aspartic acid, and the “polar sub-group” consisting of asparagine and glutamine.

Aromatic amino acids may be sub-divided into two sub-groups: the “nitrogen ring sub-group” consisting of histidine and tryptophan and the “phenyl sub-group” consisting of phenylalanine and tyrosine.

The amino acid replacement or substitution can be conservative, semi-conservative, or non-conservative. The phrase “conservative amino acid substitution” or “conservative mutation” refers to the replacement of one amino acid by another amino acid with a common property. A functional way to define common properties between individual amino acids is to analyze the normalized frequencies of amino acid changes between corresponding proteins of homologous organisms (Schulz and Schirmer, Principles of Protein Structure, Springer-Verlag, New York (1979)). According to such analyses, groups of amino acids may be defined where amino acids within a group exchange preferentially with each other, and therefore resemble each other most in their impact on the overall protein structure (Schulz and Schirmer, supra).

Examples of conservative amino acid substitutions include substitutions of amino acids within the sub-groups described above, for example, lysine for arginine and vice versa such that a positive charge may be maintained, glutamic acid for aspartic acid and vice versa such that a negative charge may be maintained, serine for threonine such that a free —OH can be maintained, and glutamine for asparagine such that a free —NH₂ can be maintained.

“Semi-conservative mutations” include amino acid substitutions of amino acids within the same groups listed above, but not within the same sub-group. For example, the substitution of aspartic acid for asparagine, or asparagine for lysine, involves amino acids within the same group, but different sub-groups. “Non-conservative mutations” involve amino acid substitutions between different groups, for example, lysine for tryptophan, or phenylalanine for serine, etc.

In addition, one or more amino acids can be inserted into the aforementioned immunoglobulin heavy chain polypeptides and/or light chain polypeptides. Any number of any suitable amino acids can be inserted into the amino acid sequence of the immunoglobulin heavy chain polypeptide and/or light chain polypeptide. In this respect, at least one amino acid (e.g., 2 or more, 5 or more, or 10 or more amino acids), but not more than 20 amino acids (e.g., 18 or less, 15 or less, or 12 or less amino acids), can be inserted into the amino acid sequence of the immunoglobulin heavy chain polypeptide and/or light chain polypeptide. Preferably, 1-10 amino acids (e.g., 1, 2, 3, 4, 5, 6, 7, 8, 9, or 10 amino acids) are inserted into the amino acid sequence of the immunoglobulin heavy chain polypeptide and/or light chain polypeptide. In this respect, the amino acid(s) can be inserted into any one of the aforementioned immunoglobulin heavy chain polypeptides and/or light chain polypeptides in any suitable location. Preferably, the amino acid(s) are inserted into a CDR (e.g., CDR1, CDR2, or CDR3) of the immunoglobulin heavy chain polypeptide and/or light chain polypeptide.

The isolated immunoglobulin heavy chain polypeptide and light chain polypeptides are not limited to polypeptides comprising the specific amino acid sequences described herein. Indeed, the immunoglobulin heavy chain polypeptide or light chain polypeptide can be any heavy chain polypeptide or light chain polypeptide that competes with the immunoglobulin heavy chain polypeptide or light chain polypeptide for binding to IL-33. In this respect, for example, the immunoglobulin heavy chain polypeptide or light chain polypeptide can be any heavy chain polypeptide or light chain polypeptide that binds to the same epitope of IL-33 recognized by the heavy and light chain polypeptides described herein. Antibody competition can be assayed using routine peptide competition assays which utilize ELISA, Western blot, or immunohistochemistry methods (see, e.g., U.S. Pat. Nos. 4,828,981 and 8,568,992; and Braitbard et al., Proteome Sci., 4: 12 (2006)).

Any amino acid residue of the immunoglobulin heavy chain polypeptide and/or the immunoglobulin light chain polypeptide can be replaced, in any combination, with a different amino acid residue, or can be deleted or inserted, so long as the biological activity of the IL-33-binding agent is enhanced or improved as a result of the amino acid replacements, insertions, and/or deletions. The “biological activity” of an IL-33-binding agent refers to, for example, binding affinity for a particular IL-33 epitope, neutralization or inhibition of IL-33 binding to its receptor(s), neutralization or inhibition of IL-33 activity in vivo (e.g., IC₅₀), pharmacokinetics, and cross-reactivity (e.g., with non-human homologs or orthologs of the IL-33 protein, or with other proteins or tissues). Other biological properties or characteristics of an antigen-binding agent recognized in the art include, for example, avidity, selectivity, solubility, folding, immunotoxicity, expression, and formulation. The aforementioned properties or characteristics can be observed, measured, and/or assessed using standard techniques including, but not limited to, ELISA, competitive ELISA, surface plasmon resonance analysis (BIACORE™), or KINEXA™, in vitro or in vivo neutralization assays, receptor-ligand binding assays, cytokine or growth factor production and/or secretion assays, and signal transduction and immunohistochemistry assays.

The IL-33-binding agent preferably inhibits or neutralizes the activity of IL-33 by at least about 20%, about 30%, about 40%, about 50%, about 60%, about 70%, about 80%, about 90%, about 95%, about 100%, or a range defined by any two of the foregoing values. The terms “inhibit” or “neutralize,” as used herein with respect to the activity of a IL-33-binding agent, refer to the ability to substantially antagonize, prohibit, prevent, restrain, slow, disrupt, alter, eliminate, stop, or reverse the progression or severity of, for example, the biological activity of IL-33, or a disease or condition associated with IL-33.

The IL-33 binding agent can be a protein (e.g., an antibody or antibody fragment) comprising, consisting essentially of, or consisting of one or more of the immunoglobulin heavy chain polypeptides and/or one or more of the immunoglobulin light chain polypeptides.

The IL-33-binding agent can be a whole antibody, as described herein, or an antibody fragment. The terms “fragment of an antibody,” “antibody fragment,” and “functional fragment of an antibody” are used interchangeably herein to mean one or more fragments of an antibody that retain the ability to specifically bind to an antigen (see, generally, Holliger et al., Nat. Biotech., 23(9): 1126-1129 (2005)). The isolated IL-33 binding agent can contain any IL-33-binding antibody fragment. The antibody fragment desirably comprises, for example, one or more CDRs, the variable region (or portions thereof), the constant region (or portions thereof), or combinations thereof. Examples of antibody fragments include, but are not limited to, (i) a Fab fragment, which is a monovalent fragment consisting of the V_{L}, V_{H}, C_{L}, and CH₁ domains, (ii) a F(ab′)₂ fragment, which is a bivalent fragment comprising two Fab fragments linked by a disulfide bridge at the hinge region, (iii) a Fv fragment consisting of the V_{L} and V_{H} domains of a single arm of an antibody, (iv) a Fab′ fragment, which results from breaking the disulfide bridge of an F(ab′)₂ fragment using mild reducing conditions, (v) a disulfide-stabilized Fv fragment (dsFv), and (vi) a domain antibody (dAb), which is an antibody single variable region domain (VH or VL) polypeptide that specifically binds antigen.

In embodiments where the IL-33-binding agent comprises a fragment of the immunoglobulin heavy chain or light chain polypeptide, the fragment can be of any size so long as the fragment binds to, and preferably inhibits the activity of, IL-33. In this respect, a fragment of the immunoglobulin heavy chain polypeptide desirably comprises between about 5 and 18 (e.g., about 5, 6, 7, 8, 9, 10, 11, 12, 13, 14, 15, 16, 17, 18, or a range defined by any two of the foregoing values) amino acids. Similarly, a fragment of the immunoglobulin light chain polypeptide desirably comprises between about 5 and 18 (e.g., about 5, 6, 7, 8, 9, 10, 11, 12, 13, 14, 15, 16, 17, 18, or a range defined by any two of the foregoing values) amino acids.

When the IL-33-binding agent is an antibody or antibody fragment, the antibody or antibody fragment desirably comprises a heavy chain constant region (F_{c}) of any suitable class. Preferably, the antibody or antibody fragment comprises a heavy chain constant region that is based upon wild-type IgG1, IgG2, or IgG4 antibodies, or variants thereof.

The IL-33-binding agent also can be a single chain antibody fragment. Examples of single chain antibody fragments include, but are not limited to, (i) a single chain Fv (scFv), which is a monovalent molecule consisting of the two domains of the Fv fragment (i.e., V_{L} and V_{H}) joined by a synthetic linker which enables the two domains to be synthesized as a single polypeptide chain (see, e.g., Bird et al., Science, 242: 423-426 (1988); Huston et al., Proc. Natl. Acad. Sci. USA, 85: 5879-5883 (1988); and Osbourn et al., Nat. Biotechnol., 16: 778 (1998)) and (ii) a diabody, which is a dimer of polypeptide chains, wherein each polypeptide chain comprises a V_{H} connected to a V_{L} by a peptide linker that is too short to allow pairing between the V_{H} and V_{L} on the same polypeptide chain, thereby driving the pairing between the complementary domains on different V_{H}-V_{L} polypeptide chains to generate a dimeric molecule having two functional antigen binding sites. Antibody fragments are known in the art and are described in more detail in, e.g., U.S. Patent Application Publication 2009/0093024 A1.

The IL-33-binding agent also can be an intrabody or fragment thereof. An intrabody is an antibody which is expressed and which functions intracellularly. Intrabodies typically lack disulfide bonds and are capable of modulating the expression or activity of target genes through their specific binding activity. Intrabodies include single domain fragments such as isolated V_{H} and V_{L} domains and scFvs. An intrabody can include sub-cellular trafficking signals attached to the N or C terminus of the intrabody to allow expression at high concentrations in the sub-cellular compartments where a target protein is located. Upon interaction with a target gene, an intrabody modulates target protein function and/or achieves phenotypic/functional knockout by mechanisms such as accelerating target protein degradation and sequestering the target protein in a non-physiological sub-cellular compartment. Other mechanisms of intrabody-mediated gene inactivation can depend on the epitope to which the intrabody is directed, such as binding to the catalytic site on a target protein or to epitopes that are involved in protein-protein, protein-DNA, or protein-RNA interactions.

The IL-33-binding agent also can be an antibody conjugate. In this respect, the isolated IL-33-binding agent can be a conjugate of (1) an antibody, an alternative scaffold, or fragments thereof, and (2) a protein or non-protein moiety comprising the IL-33-binding agent. For example, the IL-33-binding agent can be all or part of an antibody conjugated to a peptide, a fluorescent molecule, or a chemotherapeutic agent.

The IL-33-binding agent can be, or can be obtained from, a human antibody, a non-human antibody, or a chimeric antibody. By “chimeric” is meant an antibody or fragment thereof comprising both human and non-human regions. Preferably, the isolated IL-33-binding agent is a humanized antibody. A “humanized” antibody is a monoclonal antibody comprising a human antibody scaffold and at least one CDR obtained or derived from a non-human antibody. Non-human antibodies include antibodies isolated from any non-human animal, such as, for example, a rodent (e.g., a mouse or rat). A humanized antibody can comprise, one, two, or three CDRs obtained or derived from a non-human antibody. In one embodiment of the invention, CDRH3 of the IL-33-binding agent is obtained or derived from a mouse monoclonal antibody, while the remaining variable regions and constant region of the IL-33-binding agent are obtained or derived from a human monoclonal antibody.

A human antibody, a non-human antibody, a chimeric antibody, or a humanized antibody can be obtained by any means, including via in vitro sources (e.g., a hybridoma or a cell line producing an antibody recombinantly) and in vivo sources (e.g., rodents). Methods for generating antibodies are known in the art and are described in, for example, Köhler and Milstein, Eur. J. Immunol., 5: 511-519 (1976); Harlow and Lane (eds.), Antibodies: A Laboratory Manual, CSH Press (1988); and Janeway et al. (eds.), Immunobiology, 5th Ed., Garland Publishing, New York, N.Y. (2001)). In certain embodiments, a human antibody or a chimeric antibody can be generated using a transgenic animal (e.g., a mouse) wherein one or more endogenous immunoglobulin genes are replaced with one or more human immunoglobulin genes. Examples of transgenic mice wherein endogenous antibody genes are effectively replaced with human antibody genes include, but are not limited to, the Medarex HUMAB-MOUSE™, the Kirin TC MOUSE™, and the Kyowa Kirin KM-MOUSE™ (see, e.g., Lonberg, Nat. Biotechnol., 23(9): 1117-25 (2005), and Lonberg, Handb. Exp. Pharmacol., 181: 69-97 (2008)). A humanized antibody can be generated using any suitable method known in the art (see, e.g., An, Z. (ed.), Therapeutic Monoclonal Antibodies: From Bench to Clinic, John Wiley & Sons, Inc., Hoboken, N.J. (2009)), including, e.g., grafting of non-human CDRs onto a human antibody scaffold (see, e.g., Kashmiri et al., Methods, 36(1): 25-34 (2005); and Hou et al., J. Biochem., 144(1): 115-120 (2008)). In one embodiment, a humanized antibody can be produced using the methods described in, e.g., U.S. Patent Application Publication 2011/0287485 A1.

In one embodiment, a CDR (e.g., CDR1, CDR2, or CDR3) or a variable region of the immunoglobulin heavy chain polypeptide and/or the immunoglobulin light chain polypeptide described herein can be transplanted (i.e., grafted) into another molecule, such as an antibody or non-antibody polypeptide, using either protein chemistry or recombinant DNA technology. The IL-33-binding agent can comprise at least one CDR of an immunoglobulin heavy chain and/or light chain polypeptide as described herein. The isolated IL-33-binding agent can comprise one, two, or three CDRs of an immunoglobulin heavy chain and/or light chain variable region as described herein. For example, with respect to immunoglobulin heavy chain polypeptides comprising any one of SEQ ID NO: 1, SEQ ID NO: 2, or SEQ ID NOs: 5-50, the CDR1 is located between amino acid residues 26 and 35, inclusive; the CDR2 is located between amino acid residues 50 and 59, inclusive (SEQ ID NO: 1 and SEQ ID NO: 2) or between amino acid residues 50 and 66, inclusive (SEQ ID NOs: 5-50); and the CDR3 is located between amino acid residues 99 and 102, inclusive (SEQ ID NO: 1 and SEQ ID NO: 2) or between amino acid residues 99 and 111, inclusive (SEQ ID NOs 5-50). With respect to immunoglobulin light chain polypeptides comprising any one of SEQ ID NO: 3, SEQ ID NO: 4, and SEQ ID NO: 51-66, for example, the CDR1 is located between amino acid residues 24 and 39, inclusive (SEQ ID NO: 3 and SEQ ID NO: 4) or between amino acid residues 24 and 34, inclusive (SEQ ID NOs: 51-66); the CDR2 is located between amino acid residues 55 and 61, inclusive (SEQ ID NO: 3 and SEQ ID NO: 4) or between amino acid residues 50 and 56, inclusive (SEQ ID NOs: 51-66); the CDR3 is located between amino acid residues 94 and 102, inclusive (SEQ ID NO: 3 and SEQ ID NO: 4) or between amino acid residues 89 and 97, inclusive (SEQ ID NOs: 51-66).

The term “nucleic acid sequence” is intended to encompass a polymer of DNA or RNA, i.e., a polynucleotide, which can be single-stranded or double-stranded and which can contain non-natural or altered nucleotides. The terms “nucleic acid” and “polynucleotide” as used herein refer to a polymeric form of nucleotides of any length, either ribonucleotides (RNA) or deoxyribonucleotides (DNA). These terms refer to the primary structure of the molecule, and thus include double- and single-stranded DNA, and double- and single-stranded RNA. The terms include, as equivalents, analogs of either RNA or DNA made from nucleotide analogs and modified polynucleotides such as, though not limited to, methylated and/or capped polynucleotides. Nucleic acids are typically linked via phosphate bonds to form nucleic acid sequences or polynucleotides, though many other linkages are known in the art (e.g., phosphorothioates, boranophosphates, and the like).

The IL-33 binding agent comprising one or more immunoglobulin heavy and/or light chains described herein can be provided using a nucleic acid encoding the polypeptides, optionally in a vector. The vector can be, for example, a plasmid, episome, cosmid, viral vector (e.g., retroviral or adenoviral), or phage. Suitable vectors and methods of vector preparation are well known in the art (see, e.g., Sambrook et al., Molecular Cloning, a Laboratory Manual, 3rd edition, Cold Spring Harbor Press, Cold Spring Harbor, N.Y. (2001), and Ausubel et al., Current Protocols in Molecular Biology, Greene Publishing Associates and John Wiley & Sons, New York, N.Y. (1994)).

In addition to the nucleic acid sequence encoding the immunoglobulin heavy polypeptide, the immunoglobulin light chain polypeptide, and/or the IL-33-binding agent, the vector preferably comprises expression control sequences, such as promoters, enhancers, polyadenylation signals, transcription terminators, internal ribosome entry sites (IRES), and the like, that provide for the expression of the coding sequence in a host cell. Exemplary expression control sequences are known in the art and described in, for example, Goeddel, Gene Expression Technology: Methods in Enzymology, Vol. 185, Academic Press, San Diego, Calif. (1990).

A large number of promoters, including constitutive, inducible, and repressible promoters, from a variety of different sources are well known in the art. Representative sources of promoters include for example, virus, mammal, insect, plant, yeast, and bacteria, and suitable promoters from these sources are readily available, or can be made synthetically, based on sequences publicly available, for example, from depositories such as the ATCC as well as other commercial or individual sources. Promoters can be unidirectional (i.e., initiate transcription in one direction) or bi-directional (i.e., initiate transcription in either a 3′ or 5′ direction). Non-limiting examples of promoters include, for example, the T7 bacterial expression system, pBAD (araA) bacterial expression system, the cytomegalovirus (CMV) promoter, the SV40 promoter, the RSV promoter. Inducible promoters include, for example, the Tet system (U.S. Pat. Nos. 5,464,758 and 5,814,618), the Ecdysone inducible system (No et al., Proc. Natl. Acad. Sci., 93: 3346-3351 (1996)), the T-REX™ system (Invitrogen, Carlsbad, Calif.), LACSWITCH™ system (Stratagene, San Diego, Calif.), and the Cre-ERT tamoxifen inducible recombinase system (Indra et al., Nuc. Acid. Res., 27: 4324-4327 (1999); Nuc. Acid. Res., 28: e99 (2000); U.S. Pat. No. 7,112,715; and Kramer & Fussenegger, Methods Mol. Biol., 308: 123-144 (2005)).

The term “enhancer” as used herein, refers to a DNA sequence that increases transcription of, for example, a nucleic acid sequence to which it is operably linked. Enhancers can be located many kilobases away from the coding region of the nucleic acid sequence and can mediate the binding of regulatory factors, patterns of DNA methylation, or changes in DNA structure. A large number of enhancers from a variety of different sources are well known in the art and are available as or within cloned polynucleotides (from, e.g., depositories such as the ATCC as well as other commercial or individual sources). A number of polynucleotides comprising promoters (such as the commonly-used CMV promoter) also comprise enhancer sequences. Enhancers can be located upstream, within, or downstream of coding sequences.

The vector also can comprise a “selectable marker gene.” The term “selectable marker gene,” as used herein, refers to a nucleic acid sequence that allow cells expressing the nucleic acid sequence to be specifically selected for or against, in the presence of a corresponding selective agent. Suitable selectable marker genes are known in the art and described in, e.g., International Patent Application Publications WO 1992/008796 and WO 1994/028143; Wigler et al., Proc. Natl. Acad. Sci. USA, 77: 3567-3570 (1980); O′Hare et al., Proc. Natl. Acad. Sci. USA, 78: 1527-1531 (1981); Mulligan & Berg, Proc. Natl. Acad. Sci. USA, 78: 2072-2076 (1981); Colberre-Garapin et al., J. Mol. Biol., 150: 1-14 (1981); Santerre et al., Gene, 30: 147-156 (1984); Kent et al., Science, 237: 901-903 (1987); Wigler et al., Cell, 11: 223-232 (1977); Szybalska & Szybalski, Proc. Natl. Acad. Sci. USA, 48: 2026-2034 (1962); Lowy et al., Cell, 22: 817-823 (1980); and U.S. Pat. Nos. 5,122,464 and 5,770,359.

In some embodiments, the vector is an “episomal expression vector” or “episome,” which is able to replicate in a host cell, and persists as an extrachromosomal segment of DNA within the host cell in the presence of appropriate selective pressure (see, e.g., Conese et al., Gene Therapy, 11: 1735-1742 (2004)). Representative commercially available episomal expression vectors include, but are not limited to, episomal plasmids that utilize Epstein Barr Nuclear Antigen 1 (EBNA1) and the Epstein Barr Virus (EBV) origin of replication (oriP). The vectors pREP4, pCEP4, pREP7, and pcDNA3.1 from Invitrogen (Carlsbad, Calif.) and pBK-CMV from Stratagene (La Jolla, Calif.) represent non-limiting examples of an episomal vector that uses T-antigen and the SV40 origin of replication in lieu of EBNA1 and oriP.

Other suitable vectors include integrating expression vectors, which may randomly integrate into the host cell's DNA, or may include a recombination site to enable the specific recombination between the expression vector and the host cell's chromosome. Such integrating expression vectors may utilize the endogenous expression control sequences of the host cell's chromosomes to effect expression of the desired protein. Examples of vectors that integrate in a site specific manner include, for example, components of the flp-in system from Invitrogen (Carlsbad, Calif.) (e.g., pcDNA™5/FRT), or the cre-lox system, such as can be found in the pExchange-6 Core Vectors from Stratagene (La Jolla, Calif.). Examples of vectors that randomly integrate into host cell chromosomes include, for example, pcDNA3.1 (when introduced in the absence of T-antigen) from Life Technologies (Carlsbad, Calif.), UCOE from Millipore (Billerica, Mass.), and pCI or pFN10A (ACT) FLEXI™ from Promega (Madison, Wis.).

Viral vectors also can be used. Representative commercially available viral expression vectors include, but are not limited to, the adenovirus-based Per.C6 system available from Crucell, Inc. (Leiden, The Netherlands), the lentiviral-based pLP1 from Invitrogen (Carlsbad, Calif.), and the retroviral vectors pFB-ERV plus pCFB-EGSH from Stratagene (La Jolla, Calif.).

Nucleic acid sequences encoding the amino acid sequences described herein can be provided to a cell on the same vector (i.e., in cis). A unidirectional promoter can be used to control expression of each nucleic acid sequence. In another embodiment, a combination of bidirectional and unidirectional promoters can be used to control expression of multiple nucleic acid sequences. Nucleic acid sequences encoding the amino acid sequences described herein alternatively can be provided to the population of cells on separate vectors (i.e., in trans). Each of the nucleic acid sequences in each of the separate vectors can comprise the same or different expression control sequences. The separate vectors can be provided to cells simultaneously or sequentially.

The vector(s) comprising the nucleic acid(s) encoding the amino acid sequences described herein can be introduced into a host cell that is capable of expressing the polypeptides encoded thereby, including any suitable prokaryotic or eukaryotic cell. Preferred host cells are those that can be easily and reliably grown, have reasonably fast growth rates, have well characterized expression systems, and can be transformed or transfected easily and efficiently.

Examples of suitable prokaryotic cells include, but are not limited to, cells from the genera Bacillus (such as Bacillus subtilis and Bacillus brevis), Escherichia (such as E. coli), Pseudomonas, Streptomyces, Salmonella, and Erwinia. Particularly useful prokaryotic cells include the various strains of Escherichia coli (e.g., K12, HB101 (ATCC No. 33694), DH5α, DH10, MC1061 (ATCC No. 53338), and CC102).

Preferably, the vector is introduced into a eukaryotic cell. Suitable eukaryotic cells are known in the art and include, for example, yeast cells, insect cells, and mammalian cells. Examples of suitable yeast cells include those from the genera Kluyveromyces, Pichia, Rhino-sporidium, Saccharomyces, and Schizosaccharomyces. Preferred yeast cells include, for example, Saccharomyces cerivisae and Pichia pastoris.

Suitable insect cells are described in, for example, Kitts et al., Biotechniques, 14: 810-817 (1993); Lucklow, Curr. Opin. Biotechnol., 4: 564-572 (1993); and Lucklow et al., J. Virol., 67: 4566-4579 (1993). Preferred insect cells include Sf-9 and HI5 (Invitrogen, Carlsbad, Calif.).

Preferably, mammalian cells are utilized. A number of suitable mammalian host cells are known in the art, and many are available from the American Type Culture Collection (ATCC, Manassas, Va.). Examples of suitable mammalian cells include, but are not limited to, Chinese hamster ovary cells (CHO) (ATCC No. CCL61), CHO DHFR-cells (Urlaub et al., Proc. Natl. Acad. Sci. USA, 97: 4216-4220 (1980)), human embryonic kidney (HEK) 293 or 293T cells (ATCC No. CRL1573), and 3T3 cells (ATCC No. CCL92). Other suitable mammalian cell lines are the monkey COS-1 (ATCC No. CRL1650) and COS-7 cell lines (ATCC No. CRL1651), as well as the CV-1 cell line (ATCC No. CCL70). Further exemplary mammalian host cells include primate cell lines and rodent cell lines, including transformed cell lines. Normal diploid cells, cell strains derived from in vitro culture of primary tissue, as well as primary explants, are also suitable. Other suitable mammalian cell lines include, but are not limited to, mouse neuroblastoma N2A cells, HeLa, mouse L-929 cells, and BHK or HaK hamster cell lines, all of which are available from the ATCC. Methods for selecting suitable mammalian host cells and methods for transformation, culture, amplification, screening, and purification of cells are known in the art.

Most preferably, the mammalian cell is a human cell. For example, the mammalian cell can be a human lymphoid or lymphoid derived cell line, such as a cell line of pre-B lymphocyte origin. Examples of human lymphoid cells lines include, without limitation, RAMOS (CRL-1596), Daudi (CCL-213), EB-3 (CCL-85), DT40 (CRL-2111), 18-81 (Jack et al., Proc. Natl. Acad. Sci. USA, 85: 1581-1585 (1988)), Raji cells (CCL-86), and derivatives thereof.

A nucleic acid sequence encoding the amino acid sequence may be introduced into a cell by “transfection,” “transformation,” or “transduction.” “Transfection,” “transformation,” or “transduction,” as used herein, refer to the introduction of one or more exogenous polynucleotides into a host cell by using physical or chemical methods. Many transfection techniques are known in the art and include, for example, calcium phosphate DNA co-precipitation (see, e.g., Murray E. J. (ed.), Methods in Molecular Biology, Vol. 7, Gene Transfer and Expression Protocols, Humana Press (1991)); DEAE-dextran; electroporation; cationic liposome-mediated transfection; tungsten particle-facilitated microparticle bombardment (Johnston, Nature, 346: 776-777 (1990)); and strontium phosphate DNA co-precipitation (Brash et al., Mol. Cell Biol., 7: 2031-2034 (1987)). Phage or viral vectors can be introduced into host cells, after growth of infectious particles in suitable packaging cells, many of which are commercially available.

The IL-33 inhibitor may be administered as part of a composition. Preferably, the composition is a pharmaceutically acceptable (e.g., physiologically acceptable) composition, and comprises a carrier, preferably a pharmaceutically acceptable (e.g., physiologically acceptable) carrier. Any suitable carrier can be used within the context of the invention, and such carriers are well known in the art. The choice of carrier will be determined, in part, by the particular site to which the composition may be administered and the particular method used to administer the composition. The composition optionally can be sterile. The composition can be frozen or lyophilized for storage and reconstituted in a suitable sterile carrier prior to use. The compositions can be generated in accordance with conventional techniques described in, e.g., Remington: The Science and Practice of Pharmacy, 21st Edition, Lippincott Williams & Wilkins, Philadelphia, Pa. (2001).

As used herein, the terms “treatment,” “treating,” and the like refer to obtaining a desired pharmacologic and/or physiologic effect, e.g., inhibiting or preventing an allergic reaction. Preferably, the effect is therapeutic, i.e., the effect partially or completely cures a disease and/or adverse symptom attributable to the disease. To this end, the inventive method comprises administering a “therapeutically effective amount” of the IL-33 inhibitor. A “therapeutically effective amount” refers to an amount effective, at dosages and for periods of time necessary, to achieve a desired therapeutic result. The therapeutically effective amount may vary according to factors such as the disease state, age, sex, and weight of the individual, and the ability of the IL-33 inhibitor to elicit a desired response in the individual. For example, a therapeutically effective amount of a composition is an amount which decreases IL-33 bioactivity in a mammal or human.

Alternatively, the pharmacologic and/or physiologic effect may be prophylactic, i.e., the effect completely or partially prevents a disease or symptom thereof. In this respect, the inventive method comprises administering a “prophylactically effective amount” of the IL-33 inhibitor. A “prophylactically effective amount” refers to an amount effective, at dosages and for periods of time necessary, to achieve a desired prophylactic result (e.g., prevention of disease onset).

A typical dose can be, for example, in the range of 1 pg/kg to 20 mg/kg of animal or human body weight; however, doses below or above this exemplary range are within the scope of the invention. The daily parenteral dose can be about 0.00001 μg/kg to about 20 mg/kg of total body weight (e.g., about 0.001 μg/kg, about 0.1 μg/kg, about 1 μg/kg, about 5 μg/kg, about 10 μg/kg, about 100 μg/kg, about 500 μg/kg, about 1 mg/kg, about 5 mg/kg, about 10 mg/kg, or a range defined by any two of the foregoing values), preferably from about 0.1 μg/kg to about 10 mg/kg of total body weight (e.g., about 0.5 μg/kg, about 1 μg/kg, about 50 μg/kg, about 150 μg/kg, about 300 μg/kg, about 750 μg/kg, about 1.5 mg/kg, about 5 mg/kg, or a range defined by any two of the foregoing values), more preferably from about 1 μg/kg to 5 mg/kg of total body weight (e.g., about 3 μg/kg, about 15 μg/kg, about 75 μg/kg, about 300 μg/kg, about 900 μg/kg, about 2 mg/kg, about 4 mg/kg, or a range defined by any two of the foregoing values), and even more preferably from about 0.5 to 15 mg/kg body weight per day (e.g., about 1 mg/kg, about 2.5 mg/kg, about 3 mg/kg, about 6 mg/kg, about 9 mg/kg, about 11 mg/kg, about 13 mg/kg, or a range defined by any two of the foregoing values). Therapeutic or prophylactic efficacy can be monitored by periodic assessment of treated patients. For repeated administrations over several days or longer, depending on the condition, the treatment can be repeated until a desired suppression of disease symptoms occurs. However, other dosage regimens may be useful and are within the scope of the invention. The desired dosage can be delivered by a single bolus administration of the composition, by multiple bolus administrations of the composition, or by continuous infusion administration of the composition.

The IL-33 inhibitor can be administered to a mammal using standard administration techniques, including oral, intravenous, intraperitoneal, subcutaneous, pulmonary, transdermal, intramuscular, intranasal, buccal, sublingual, or suppository administration. The composition preferably is suitable for parenteral administration. The term “parenteral,” as used herein, includes intravenous, intramuscular, subcutaneous, rectal, vaginal, and intraperitoneal administration. More preferably, the composition is administered to a mammal using peripheral systemic delivery by intravenous, intraperitoneal, or subcutaneous injection.

The IL-33 inhibitor also can be administered by introducing a nucleic acid encoding the IL-33 inhibitor to the mammal, whereby the IL-33 inhibitor is expressed in the mammal. The nucleic acid encoding the IL-33 inhibitor can be in a vector, as described herein with respect to other embodiments. Furthermore, the nucleic acid encoding the IL-33 inhibitor can be administered directly to the mammal, or administered to a cell (e.g., an autologous cell) to provide a transformed cell that expresses the IL-33 inhibitor, and the transformed cell can then be administered to the mammal. In addition, or alternatively, IL-33 inhibition may be achieved by introduction or deletion of genetic material that modulates the expression of IL-33. Techniques for administering nucleic acids to mammals and cells to express proteins, techniques for transforming cells and administering transformed cells to mammals, and techniques for deleting genetic material are known in the art.

Once administered to a mammal (e.g., a cross-reactive human), the biological activity of the IL-33 inhibitor can be measured by any suitable method known in the art. For example, the biological activity can be assessed by determining the stability of a particular IL-33 inhibitor. In one embodiment of the invention, the IL-33 inhibitor (e.g., an antibody) has an in vivo half life between about 30 minutes and 45 days (e.g., about 30 minutes, about 45 minutes, about 1 hour, about 2 hours, about 4 hours, about 6 hours, about 10 hours, about 12 hours, about 1 day, about 5 days, about 10 days, about 15 days, about 25 days, about 35 days, about 40 days, about 45 days, or a range defined by any two of the foregoing values). In another embodiment, the IL-33 inhibitor has an in vivo half life between about 2 hours and 20 days (e.g., about 5 hours, about 10 hours, about 15 hours, about 20 hours, about 2 days, about 3 days, about 7 days, about 12 days, about 14 days, about 17 days, about 19 days, or a range defined by any two of the foregoing values). In another embodiment, the IL-33 inhibitor has an in vivo half life between about 10 days and about 40 days (e.g., about 10 days, about 13 days, about 16 days, about 18 days, about 20 days, about 23 days, about 26 days, about 29 days, about 30 days, about 33 days, about 37 days, about 38 days, about 39 days, about 40 days, or a range defined by any two of the foregoing values).

The biological activity of a particular IL-33-binding agent also can be assessed by determining its binding affinity to IL-33 or an epitope thereof. The term “affinity” refers to the equilibrium constant for the reversible binding of two agents and is expressed as the dissociation constant (K_{D}). Affinity of a binding agent to a ligand, such as affinity of an antibody for an epitope, can be, for example, from about 1 femtomolar (fM) to about 100 micromolar (□M) (e.g., from about 1 fM to about 1 picomolar (pM), from about 1 pM to about 1 nanomolar (nM), from about 1 nM to about 1 micromolar (□M), or from about 1 □M to about 100 □M). In one embodiment, the IL-33-binding agent can bind to an IL-33 protein with a K_{D} less than or equal to 1 nanomolar (e.g., 0.9 nM, 0.8 nM, 0.7 nM, 0.6 nM, 0.5 nM, 0.4 nM, 0.3 nM, 0.2 nM, 0.1 nM, 0.05 nM, 0.025 nM, 0.01 nM, 0.001 nM, or a range defined by any two of the foregoing values). In another embodiment, the IL-33-binding agent can bind to IL-33 with a K_{D} less than or equal to 200 pM (e.g., 190 pM, 175 pM, 150 pM, 125 pM, 110 pM, 100 pM, 90 pM, 80 pM, 75 pM, 60 pM, 50 pM, 40 pM, 30 pM, 25 pM, 20 pM, 15 pM, 10 pM, 5 pM, 1 pM, or a range defined by any two of the foregoing values). Immunoglobulin affinity for an antigen or epitope of interest can be measured using any art-recognized assay. Such methods include, for example, fluorescence activated cell sorting (FACS), separable beads (e.g., magnetic beads), surface plasmon resonance (SPR), solution phase competition (KINEXA™), antigen panning, and/or ELISA (see, e.g., Janeway et al. (eds.), Immunobiology, 5th ed., Garland Publishing, New York, N.Y., 2001).

In one embodiment of the invention, there is provided a method of prophylactically treating a patient previously diagnosed with an allergic reaction, including but not limited to an allergic response to peanut, comprising administering to the patient an IL-33 inhibitor prior to exposure to allergen (e.g., peanut allergen) or prior onset of allergic reaction (e.g., allergic reaction to peanut allergen). In other words, according to this embodiment, the IL-33 inhibitor is administered to a patient with a known allergy when the patient is not exhibiting an allergic response. The method can further comprise regularly treating the patient with an anti-IL-33 inhibitor, irrespective of the occurrence of peanut allergy symptoms between treatments, as a prophylactic therapy to mitigate allergic response to peanut upon an unexpected exposure to peanut allergen. Thus, “prophylactic” treatment in this context does not require complete prevention, but only lessening of the severity of the allergic reaction or symptom thereof to some degree. Any suitable frequency of IL-33 inhibitor administration can be used, such as daily, weekly, biweekly, monthly, bimonthly or trimonthly. Upon any exposure of such a patient to the allergen (e.g., peanut allergen), the presence of an IL-33 inhibitor within the bloodstream and/or tissues of the patient prior to exposure to peanut is intended to suppress the allergic response, and potential anaphylactic shock, upon such exposure.

In another embodiment, the method comprises administerting an IL-33 inhibitor to a patient previously diagnosed with an allergic reaction, including but not limited to an allergic response to peanut, at such time when exposure to peanut allergen is a potential occurrence, or once exposure to peanut allergen is believed to have occurred or has actually occurred, but prior to the onset of anaphylactic shock (e.g., prior to the clinical manifestation of anaphylactic shock). The determination of the onset of anaphylactic shock is within the skill of the ordinary medical practitioner, and includes, for instance, the onset of symptoms including impaired breathing, swelling in the throat, a sudden drop in blood pressure, pale skin or blue lips, fainting, and/or dizziness, following exposure to an allergen. Administration of the IL-33 inhibitor can be self-administration. For instance, the patient can carry an anti-IL-33 inhibitor treatment with him or her for on-demand administration as needed, potentially in a situation where peanut allergen exposure is anticipated as a potential risk, or once exposure is believed to have potentially already occurred. Thus, the an IL-33 inhibitor in an auto injection device, such as a spring-loaded auto injector, that automatically inserts the needle into the skin of the patient and administers the drug through the needle upon pressing a button or pressing the device against the skin.

The IL-33 inhibitor may be administered alone or in combination with other drugs (e.g., as an adjuvant). For example, other agents for the treatment or prevention of allergic reactions can be used. Such agents include antihistamines, other anti-inflammatory agents, such as corticosteroids (e.g., prednisone and fluticasone) and non-steroidal anti-inflammatory drugs (NSAIDs) (e.g., aspirin, ibuprofen, and naproxen).

The following examples are intended to illustrate the invention, but do not limit the scope of the invention otherwise described.

### EXAMPLE 1

An anti-IL-33 antibody that inhibits IL-33 signalling, with functional characteristcs indicated above, was generated and tested in multiple dose GLP compliant toxicology studies in cynomolgus monkeys over periods of 4 and 13 weeks, following in each case with an 8 week recovery period. Results show the IL-33 neutralizing agent was well-tolerated in cynomolgus monkeys, with no unscheduled sacrifice or significant treatment-related effects. There were no concerns on safety pharmacology parameters: CNS, cardiovascular and respiratory function as evaluated in this study. No adverse effects, macroscopic or histopathological after administration of the neutralzing agent at any dose level tested.

### EXAMPLE 2

A neutralizing anti-IL-33 antibody as described herein was tested in a first-in-human, single ascending dose and multiple asceding dose, Phase 1 clinical study. In total, seventy-two healthy volunteers were dosed with the IL-33 neutralizing agent at a wide dose range of 10 mg to 750 mg. No dose-limiting toxicities were observed, and the IL-33 neutralizing agent was well tolerated.

Blood samples were collected from patients at different time points, and IL-33 inhibitory activity was tested in a whole blood, ex vivo assay upon stimulation with IL-33, and inhibition of IFN-release by the IL-33 inhibitor was measured. Persistent and nearly complete inhibition was observed for up to at least 3 months.

### EXAMPLE 3

The data generated in the GLP toxicology study and the data from the Phase 1 clinical study described in Examples 1 and 2 has supported the initiation of Phase 2 studies in patients with atopic disorders such a as peanut allergy. The therapeutic activity of an IL-33 inhibitor will be tested in adult patients with peanut allergy. At a screening visit each eligible patient will be administered a graded peanut and placebo oral food challenge (BPCFC) according to the PRACTALL consensus report. Subjective symptoms during the BPCFC will be monitored per PRACTALL guidelines using the oral food challenge (OFC) Symptom Scoring Assessment Tool. The total cumulative dose of blinded peanut/placebo tolerated and dosing step/threshold reached prior to the reaction will be recorded. After this first BPCFC, eligible patients will be randomized to receive either IL-33 inhibitor or placebo. Patients will then receive a second peanut and placebo oral food challenge according to the PRACTALL guidelines. Symptoms will be monitored using the OFC Symptom Scoring Assessment Tool and other safety assessments will be performed. The total cumulative dose of blinded peanut/placebo tolerated and dosing step/threshold reached prior to reaction will be recorded. Change from baseline, first BPCFC, tolerated dose and OFC scores will be compared between IL-33 inhibitor and placebo using mixed-effect analysis of covariance (ANCOVA). Improved tolerability in the IL-33 inhbitor treated patients compared to placebo, according to the PRACTALL guidelines, will determine activity of IL-33 inhibition in peanut allergic patients.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of inhibiting or preventing an allergic reaction in a mammal comprising administering to the mammal an IL-33 inhibitor.

2. The method of claim 1, wherein the allergic reaction is a food allergy.

3. The method of claim 1, wherein the allergic reaction is asthma, atopic dermatitis, or a combination thereof.

4. The method of claim 2, wherein the food allergy is a nut allergy, peanut allergy, shellfish allergy, fish allergy, milk allergy, egg allergy, wheat allergy, or soybean allergy.

5. The method of claim 1, wherein the IL-33 inhibitor is an anti-IL-33 antibody or antibody fragment.

6. The method of claim 5, wherein the anti-IL-33 antibody or antibody fragment comprises an isolated immunoglobulin heavy chain variable region polypeptide which comprises (a) an amino acid sequence of any one of SEQ ID NO: 1, SEQ ID NO: 2, SEQ ID NOs: 5-50, SEQ ID NOs: 67-140, SEQ ID NO: 176, SEQ ID NO: 177, SEQ ID NOs: 178-188, and SEQ ID NOs: 206-217, (b) an amino acid sequence that is at least 90% identical to any one of SEQ ID NO: 1, SEQ ID NO: 2, SEQ ID NOs: 5-50, SEQ ID NOs: 67-140, SEQ ID NO: 176, SEQ ID NO: 177, SEQ ID NOs: 178-188, and SEQ ID NOs: 206-217; or (c) an immunoglobulin heavy chain polypeptide which binds to the same IL-33 epitope as the immunoglobulin heavy chain polypeptide of (a) or (b); and
an isolated immunoglobulin light chain polypeptide which comprises (d) an amino acid sequence of any one of SEQ ID NO: 3, SEQ ID NO: 4, SEQ ID NOs: 51-66, SEQ ID NOs: 141-175, SEQ ID NOs: 189-205, and SEQ ID NOs: 218-231, (e) an amino acid sequence that is at least 90% identical to any one of SEQ ID NO: 3, SEQ ID NO: 4, SEQ ID NOs: 51-66, SEQ ID NOs: 141-175, SEQ ID NOs: 189-205, and SEQ ID NOs: 218-231; or (f) an immunoglobulin heavy chain polypeptide which binds to the same IL-33 epitope as the immunoglobulin heavy chain polypeptide of (d) or (e).

7. (canceled)

8. The method of claim 1, wherein the IL-33-inhibitor is an F(ab′)2, Fab′, Fab, Fv, scFv, dsFv, dAb, or a single chain binding polypeptide.

9. The method of claim 1, wherein the IL-33 inhibitor comprises an immunoglobulin heavy chain variable region polypeptide of SEQ ID NO: 136 and an immunoglobulin light chain variable region polypeptide of SEQ ID NO: 171.

10. The method of claim 1, wherein the isolated IL-33 inhibitor comprises heavy chain CDR1, CDR2, and CDR3 of any one of SEQ ID NO: 1, SEQ ID NO: 2, SEQ ID NOs: 5-50, SEQ ID NOs: 67-140, SEQ ID NO: 176, SEQ ID NO: 177, SEQ ID NOs: 178-188, and SEQ ID NOs: 206-217; and
light chain CDR1, CDR2, and CDR3 of any one of SEQ ID NO: 3, SEQ ID NO: 4, SEQ ID NOs: 51-66, SEQ ID NOs: 141-175, SEQ ID NOs: 189-205, and SEQ ID NOs: 218-231.

11. The method of claim 10, wherein the IL-33 inhibitor comprises heavy chain CDR1, CDR2, and CDR3 of SEQ ID NO: 136; and light chain CDR1, CDR2, and CDR3 of SEQ ID NO: 171.

12.-17. (canceled)

18. The method of claim 1, wherein the half-life of the IL-33 inhibitor in the mammal is between 30 minutes and 45 days.

19. The method of claim 1, wherein the IL-33 inhibitor binds to IL-33 with a KD between about 1 femtomolar (fM) and about 100 micromolar (μM).

20. The method of claim 1, wherein the mammal has a mutation that increases IL-33 expression or signaling.

21. The method of claim 1, where the mammal is periodically treated with an anti-IL-33 inhibitor as a prophylactic therapy.

22. The method of claim 1, where the mammal is treated with an anti-IL-33 inhibitor when exposure to an allergen has occurred or is suspected to have occurred.

23. (canceled)
